(19) United States
(12) Patent Application Publication
(10) Pub. No.: US 2026/0175017 A1
(43) Pub. Date: Jun. 25, 2026

(54) CONFIGURATIONS OF COIL FOR ENDOVASCULAR THERAPY SYSTEM

(71) Applicant: Covidien LP, Mansfield, MA (US)

(72) Inventors: Bernard Q. Li, Plymouth, MN (US); Haitao Zhang, Maple Grove, MN (US)

(21) Appl. No.: 19/427,001

(22) Filed: Dec. 19, 2025

Related U.S. Application Data
(60) Provisional application No. 63/737,107, filed on Dec. 20, 2024.

(51) Int. Cl. A61N 1/05; A61N 1/36

(52) U.S. Cl. CPC A61N 1/0534; A61N 1/0539; A61N 1/36053

(57) ABSTRACT

An endovascular therapy system includes an endovascular device. The endovascular device includes an elongated body configured to be introduced into vasculature of a patient. The endovascular device includes a coil structure at a distal portion of the elongated body. The coil structure includes at least a first coil portion and a second coil portion. The first coil portion can be configured to carry a plurality of electrodes. The second coil portion can be formed from a coiled wire such that the second coil portion together with the coiled wire defines a coiled coil. The coiled wire can extend distally of a distal end of the first coil portion and be configured to anchor the elongated body and the coil structure within the vasculature of the patient.

### CROSS-REFERENCE TO RELATED APPLICATIONS

This application claims the benefit of and priority to U.S. Provisional Patent Application Ser. No. 63/737,107 filed Dec. 20, 2024, the entire disclosure of which is incorporated by reference herein.

### TECHNICAL FIELD

This disclosure relates to electrical stimulation therapy.

### BACKGROUND

Medical devices, such as electrical stimulation devices, may be used in different therapeutic applications, such as deep brain stimulation (DBS), peripheral nerve stimulation (PNS), and vagus nerve stimulation (VNS). A medical device may be used to deliver therapy to a patient to treat a variety of symptoms or patient conditions such as, but not limited to, movement disorders, seizure disorders (e.g., epilepsy), or mood disorders. In some therapy systems, an external or an implantable electrical stimulator delivers electrical stimulation therapy to a target tissue site within a patient with the aid of one or more electrodes. Additionally or alternatively, a medical device senses one or more patient parameters with the aid of the one or more electrodes.

### SUMMARY

This disclosure describes medical devices systems (e.g., endovascular therapy systems) including endovascular devices configured for delivery of electrical stimulation therapy to a patient (e.g., to one or more nerves or brain targets) and/or sensing of one or more patient parameters (e.g., nerve signals, brain signals, and/or other physiological parameters), and related methods. In particular, this disclosure describes configurations for structures of endovascular therapy systems that facilitate delivery of electrical stimulation therapy and/or sensing of patient parameters from an endovascular location.

In examples described herein, an endovascular therapy system includes an endovascular device (e.g., which may be and/or include a medical lead) including an elongated body and one or more electrodes carried by the elongated body. The electrodes can be positioned on a portion of the endovascular device configured to deploy (e.g., expand), such as a portion that defines a coil shape (e.g., a coil portion of the elongated body).

In some cases, the elongated body (e.g., of the medical lead) can become endothelialized (e.g., become integrated with and/or into the endothelium of the blood vessel). Endothelialization can fix the position of the elongated body and/or the electrodes relative to the blood vessel and/or target tissue surrounding the blood vessel. Thus, movement of electrodes relative to target tissue occurring over relatively longer periods of time can be reduced, mitigated, or even prevented when adequate endothelialization occurs. However, prior to, and/or in the absence of, adequate endothelization in which the elongated body and/or the electrodes become incorporated into the vessel wall of the blood vessel, forces acting on the elongated body can cause undesirable movement (e.g., axial movement, rotational movement, or another types of movement).

In some cases, while the coil portion of the elongated body can be configured to anchor the elongate body and/or the electrodes within the vasculature of the patient, the anchoring force provided by a coil portion of the elongated body may not be sufficient to ensure that the electrodes remain in stable position. For example, the coil portion of the elongated body (e.g., a medical lead) may not, by itself, provide enough anchoring force within the blood vessel to reduce and/or prevents the tendency of the elongated body and/or the electrodes to move under certain conditions (e.g., relatively strong forces).

In one or more examples, the endovascular device includes and/or defines one or more structural features configured to help anchor the elongated body and/or the electrodes within the vasculature of the patient. For example, in some examples, the endovascular device can include a coil structure having at least first coil portion that carries the electrodes and a second coil portion configured to help anchor the elongated body, including the first coil portion, within the vasculature of the patient. The first coil portion can be a coil shape defined by a portion of the elongated body (e.g., of the medical lead). In some examples, the second coil portion can include and/or define one or more of a different shape, form factor, material, and/or other characteristics as compared to the first coil portion. For example, the second coil portion can include a coiled wire, e.g., such that the second coil portion together with the coiled wire defines (e.g., forms) a coiled coil. That is, the coiled wire of the second coil portion may be a continuous wire that is coiled. The coiled wire is then arranged in a coiled manner (e.g., serpentine or twisting manner) to form a coiled coil, where the coils of the wire form a coiled wire, and the serpentine or twisting of the coiled wire forms the second coil portion which is a coiled coil of wire.

In examples in which the second coil portion includes a coiled wire, the coiled wire can be configured to facilitate anchoring of the endovascular device. In some examples, the second coil portion includes a coiled wire (e.g., such that the second coil portion is a coiled coil). Using a coiled wire that forms a coil for anchoring the elongated body and the electrodes within the vasculature of the patient can facilitate relatively better anchoring, e.g., as compared other anchoring mechanisms, such as a coil portion of an elongated body (e.g., as the elongated body, which can be a portion of a medical lead, can have a relatively smooth outer surface). For example, a coiled wire may have relatively greater engagement (e.g., contact, friction, and/or the like) with a vessel wall causing the elongated body to be relatively more firmly anchored within vasculature of the patient.

In some examples, an endovascular device includes an elongated body configured to be introduced into vasculature of a patient; and a coil structure at a distal portion of the elongated body, the coil structure including: a first coil portion configured to carry a plurality of electrodes; and a second coil portion formed from a coiled wire such that the second coil portion together with the coiled wire defines a coiled coil, the coiled wire extending distally of a distal end of the first coil portion and configured to anchor the elongated body and the coil structure within the vasculature of the patient.

In some examples, a method includes introducing an endovascular device into vasculature of a patient, the endovascular device includes an elongated body configured to be introduced into the vasculature of the patient, and a coil structure at a distal portion of the elongated body, the coil structure including: a first coil portion configured to carry a plurality of electrodes, and a second coil portion formed from a coiled wire such that the second coil portion together with the coiled wire defines a coiled coil, the coiled wire extending distally of a distal end of the first coil portion and configured to anchor the elongated body and the coil structure within the vasculature of the patient; and advancing the endovascular device until the plurality of electrodes are at or near a target location in the vasculature of the patient.

In some examples, an endovascular device includes an elongated body configured to be introduced into vasculature of a patient; and a coil structure at a distal portion of the elongated body, the coil structure including: a first coil portion defining a first pitch, a first coil diameter, and a central longitudinal axis extending through a radial center of the first coil portion such that the first coil portion extends around the central longitudinal axis; a second coil portion defining a second pitch and a second coil diameter, the second coil portion formed from a coiled wire such that the second coil portion together with the coiled wire defines a coiled coil, the coiled wire extending distally of a distal end of the first coil portion and configured to anchor the elongated body and the coil structure within the vasculature of the patient; and a plurality of electrodes carried by the first coil portion such that axially adjacent electrodes of the plurality of electrodes are axially spaced apart along the central longitudinal axis at respective axial locations, wherein the coiled wire defines a third pitch, the third pitch less than the first pitch and less than the second pitch, and wherein the coiled wire defines a third coil diameter, the third coil diameter less than the first coil diameter and the second coil diameter.

The examples described herein may be combined in any permutation or combination.

The details of one or more examples of the disclosure are set forth in the accompanying drawings and the description below. Other features, objects, and advantages of the disclosure will be apparent from the description and drawings, and from the claims.

### BRIEF DESCRIPTION OF DRAWINGS

FIG. 1 is a conceptual diagram illustrating an example endovascular therapy system including an endovascular device configured for delivering electrical stimulation therapy to a target tissue site of a patient and/or sensing one or more patient parameters.

FIG. 2 is a functional block diagram illustrating components of an example endovascular device of the therapy system of FIG. 1.

FIG. 3A is a side view of an example distal portion of an example endovascular device.

FIG. 3B illustrates a cross-sectional view of the example endovascular device of FIG. 3A.

FIG. 3C is a detail view of a portion of the endovascular device of FIG. 3A.

FIG. 3D illustrates of an example endovascular device positioned within vasculature of a patient.

FIG. 4A is a side view of an example distal portion of an example endovascular device.

FIG. 4B illustrates a cross-sectional view of the example endovascular device of FIG. 4A.

FIG. 5 is a flow diagram illustrating an example technique for introducing and advancing an endovascular device according to this disclosure.

Like reference characters denote like elements throughout the description and figures.

### DETAILED DESCRIPTION

This disclosure describes devices, systems, and methods relating to delivery of electrical stimulation therapy, such as vagus nerve stimulation (VNS), deep brain stimulation (DBS), and/or sensing one or more patient parameters (e.g., nerve activity from one more nerves, cardiac signals, muscle activation signals, brain signals and/or other physiological parameters, such as impedance, electroencephalogram (EEG), evoked potentials, local field potentials, other bioelectric signals, and the like) from an endovascular location. Example endovascular locations that can be used to access the brain sites for electrical stimulation therapy (e.g., DBS) and/or sensing using the devices described herein include any suitable cranial blood vessel (also referred to herein as a cerebral blood vessel or neurovasculature, which can include a vein or an cranial artery), such as, but not limited to, the thalamostriate vein, the internal cerebral vein, the basal vein of Rosenthal, the inferior sagittal sinus, the superior sagittal sinus, or the anterior choroidal artery. Example endovascular locations that can be used for electrical stimulation therapy (e.g., VNS therapy) and/or sensing using the devices described herein include an internal jugular vein (IJV).

DBS has been proposed for use to manage one or more patient conditions, such as to treat a patient condition by reducing or even eliminating one or more symptoms associated with the patient condition. For example, DBS can be used to alleviate, and in some cases eliminate, symptoms associated with movement disorders, other neurodegenerative impairment, seizure disorders, psychiatric disorders (e.g., mood disorders), or the like. Movement disorders may be found in patients with Parkinson's disease, multiple sclerosis, and cerebral palsy, among other conditions, and can be associated with disease or trauma. DBS can be delivered to one or more target sites in a brain of a patient to help a patient with muscle control and minimize movement problems, such as rigidity, bradykinesia (i.e., slow physical movement), rhythmic hyperkinesia (e.g., tremor), nonrhythmic hyperkinesia (e.g., tics) or akinesia (i.e., a loss of physical movement).

In the case of seizure disorders, DBS can be delivered to one or more target sites in a brain of a patient to reduce the frequency or severity of seizures, or even help prevent the occurrence of seizures. In the case of psychiatric disorders, DBS can be delivered to help minimize or even eliminate symptoms associated with major depressive disorder (MDD), bipolar disorder, anxiety disorders, post-traumatic stress disorder, dysthymic disorder, or obsessive-compulsive disorder (OCD).

VNS has been proposed for use to manage one or more patient conditions, such as to control an inflammatory response in patients. Stimulating the vagus nerve may dampen the inflammatory response and associated cytokine response. In some examples, inflammatory cytokines are modulated up or down via stimulation. In addition, VNS may assist in stroke rehabilitation and limit ischemia reperfusion injury. After a myocardial infarct or stroke, reperfusion therapies (surgery or drugs) are given to restore blood flow. However, due to the restoration of blood, flow induced local damage occurs, including ischemia reperfusion injury. This injury may induce local accumulations of chemical mediators such as reactive oxygen species (ROS) production, inflammatory cytokines, bradykinin, etc., which can further affect inflammation. Such inflammatory compounds may trigger sensory signaling, which can lead to a reduced organ vagus activity and sympathetic overdrive. Vagus nerve stimulation may treat reperfusion damage as the inflammatory state may be lowered by increasing parasympathetic drive.

In examples described herein, an endovascular therapy system includes an endovascular device (e.g., which may be and/or include a medical lead) including an elongated body and one or more electrodes carried by the elongated body. Additionally or alternatively, one or more sensing elements are carried by the elongated body. Each electrode and/or sensing element is electrically connected to a medical device via one or more electrically conductive elements (e.g., conductor wires, trace elements, and/or the like). The medical device is configured to deliver therapy (e.g., electrical stimulation therapy) and/or received sensed signals via the electrodes by way of the electrically conductive elements. The electrically conductive elements can extend from the medical device, along the elongated body, and to each electrode to electrically connect to each electrode or sensing element.

In examples described herein, at least a portion of the endovascular therapy system (e.g., the elongated body and the electrodes) are configured to be positioned within vasculature of a patient (e.g., within a blood vessel) and deliver electrical stimulation therapy to target tissue (e.g., one or more nerves and/or brain tissue) located outside of (e.g., radially outside of) the blood vessel in which the electrodes are positioned. Additionally and/or alternatively, the endovascular therapy systems herein are configured to receive signals (e.g., corresponding to one or more patient parameters) from the one or more nerves and/or brain tissue located outside of the blood vessel in which the electrodes and/or sensing elements are positioned.

In examples described herein, one or more electrodes are positioned on a portion of the elongated body configured to transform between a delivery (e.g., compressed or relatively low-profile) configuration and a deployed (e.g., expanded) configuration. The elongated body can be configured to place the one or more electrodes into apposition and/or into contact with a blood vessel wall of the blood vessel in which the elongated body and the electrodes are positioned. For example, the one or more electrodes can be positioned on a portion of the elongated body configured to transform into a deployed configuration in which the elongated body defines a coil shape. In some examples, the elongated body includes a coil structure that defines a coil shape. The coil structure can be configured to position the one or more electrodes into apposition with the blood vessel wall.

In some cases, the positioning of the electrodes relative to the target location, such as relative to one or more nerves and/or brain tissue located radially outside of the blood vessel in which the electrodes are positioned, can affect the efficacy of therapy, e.g., electrical stimulation therapy, delivered to the one or more nerves and/or brain tissue. For example, electrodes can be placed at an axial location within a blood vessel where the target tissue (e.g., a target nerve and/or brain tissue) is relatively close to the blood vessel as compared to other axial locations of the blood vessel. In some cases, the closeness (e.g., physical proximity) of a nerve or other target tissue to a blood vessel changes along an axial direction of the blood vessel. In some cases, efficacy of therapy (e.g., the effect of electrical stimulation therapy) and/or the ability to sense (e.g., ability to detect electrical signals) changes with the closeness (e.g., physical proximity) of the nerve or other target tissue to the blood vessel.

In some cases, the elongated body (e.g., of the medical lead) can become endothelialized (e.g., become integrated with and/or into the endothelium of the blood vessel). Endothelialization can fix the position of the elongated body and/or the electrodes relative to the blood vessel and/or target tissue surrounding the blood vessel. Thus, movement of electrodes relative to target tissue occurring over relatively longer periods of time can be reduced, mitigated, or even prevented when adequate endothelialization occurs. However, prior to, and/or in the absence of, adequate endothelization in which the elongated body and/or the electrodes become incorporated into the vessel wall of the blood vessel, forces acting on the elongated body can cause undesirable movement (e.g., axial movement, rotational movement, or another types of movement).

In some cases, while the coil portion of the elongated body can be configured to anchor the elongate body and/or the electrodes within the vasculature of the patient, the anchoring force provided by a coil portion of the elongated body may not be sufficient to ensure that the electrodes remain in stable position. An anchoring force can include a force directed radially outward toward a blood vessel wall that reduces or prevents the tendency of the elongated body and/or the electrodes to move axially and/or rotationally with respect to the blood vessel in response to an external force. In some cases, the coil portion of the elongated body (e.g., a medical lead) may not, by itself, provide enough anchoring force within the blood vessel to reduce and/or prevents the tendency of the elongated body and/or the electrodes to move under certain conditions (e.g., relatively strong forces).

In one or more examples, the endovascular device includes and/or defines one or more structural features configured to help anchor the elongated body and/or the electrodes within the vasculature of the patient. For example, in some examples, the endovascular device can include a coil structure having at least first coil portion that carries the electrodes and a second coil portion configured to help anchor the elongated body, including the first coil portion, within the vasculature of the patient. The first coil portion can be a coil shape defined by a portion of the elongated body (e.g., of the medical lead). In some examples, the second coil portion can include and/or define one or more of a different shape, form factor, material, and/or other characteristics as compared to the first coil portion. For example, the second coil portion can include a coiled wire, e.g., such that the second coil portion together with the coiled wire defines (e.g., forms) a coiled coil. That is, the coiled wire of the second coil portion may be a continuous wire that is coiled. The coiled wire is then arranged in a coiled manner (e.g., serpentine or twisting manner) to form a coiled coil, where the coils of the wire form a coiled wire, and the serpentine or twisting of the coiled wire forms the second coil portion which is a coiled coil of wire.

In some examples, the coiled wire that forms the second coil portion is mechanically coupled to the first coil portion of the elongated body. In some examples, the second coil portion includes a coiled wire that is at least partially coextensive with the elongated body (e.g., the wire forming the coiled wire is coextensive with one or more conductor wires that extend within the elongate body).

In some examples, the first coil portion and the second coil portion define a continuous or substantially continuous coil structure. For example, the first coil portion can include a diameter and pitch (e.g., spacing) that is the same or substantially the same as a diameter and pitch of the second coil portion. The continuous and/or the substantially continuous coil shape formed by the first coil portion and the second coil portion can facilitate relatively easier transformation of the endovascular device to a relatively low-profile delivery configuration, which can facilitate and enable easier delivery and/or retrieval (e.g., re-sheathing) of the elongated body, the first coil portion and/or the second coil portion (e.g., as compared to elongated bodies having anchoring structures that do not form continuous or substantially continuous coil shapes). The relatively simple, continuous shape of the coil formed by the first coil portion and the second coil portion (e.g., as compared to other expandable anchoring structures, such as stents and/or stent-like structures) can also enable relatively simpler or easier manufacturing and/or assembly.

In examples in which the second coil portion includes a coiled wire, the coiled wire can be configured to facilitate anchoring of the endovascular device. In some examples, the second coil portion includes a coiled wire (e.g., such that the second coil portion is a coiled coil). Using a coiled wire that forms a coil for anchoring the elongated body and the electrodes within the vasculature of the patient can facilitate relatively better anchoring, e.g., as compared other anchoring mechanisms, such as a coil portion of an elongated body (e.g., as the elongated body, which can be a portion of a medical lead, can have a relatively smooth outer surface). For example, a coiled wire may have relatively greater engagement (e.g., contact, friction, and/or the like) with a vessel wall causing the elongated body to be relatively more firmly anchored within vasculature of the patient.

In some examples, a medical device is configured to generate electrical stimulation and/or sense a patient parameter via the electrodes of the endovascular device. For example, once the electrodes are positioned into apposition with a blood vessel wall, the medical device can be configured to generate and delivery electrical stimulation via the electrodes to target tissue (e.g., nerves and/or brain tissue located radially outside of the blood vessel). As another example, the medical device can be configured to receive signals (e.g., electrical signals corresponding to a patient parameter) via the electrodes from the target tissue (e.g., nerves and/or brain tissue located radially outside of the blood vessel).

While one or more examples of this disclosure describe placement of endovascular devices within the neurovasculature, it should be appreciated that the devices and techniques of this disclosure can be configured for placement in other anatomical locations and/or anatomical structures. For example, the devices and techniques described herein may be configured for placement in one or more of a dural sinus, vein, or artery in close proximity to a target area in the brain, but may also be adapted and configured for placement in other locations within a patient (e.g., the peripheral vasculature) in proximity to one or more peripheral nerves, a nerve plexus, and/or other nervus system targets for electrical stimulation therapy and/or sensing. In some examples, the systems, devices, and method described herein can be adapted for stimulation and/or sensing of a vagus nerve (e.g., from a location from within a suitable blood vessel, including a jugular vein and/or a carotid artery). In some examples, the devices and techniques of this disclosure may also be adapted and configured for placement within the ventricles or other hollow anatomical structures of the brain.

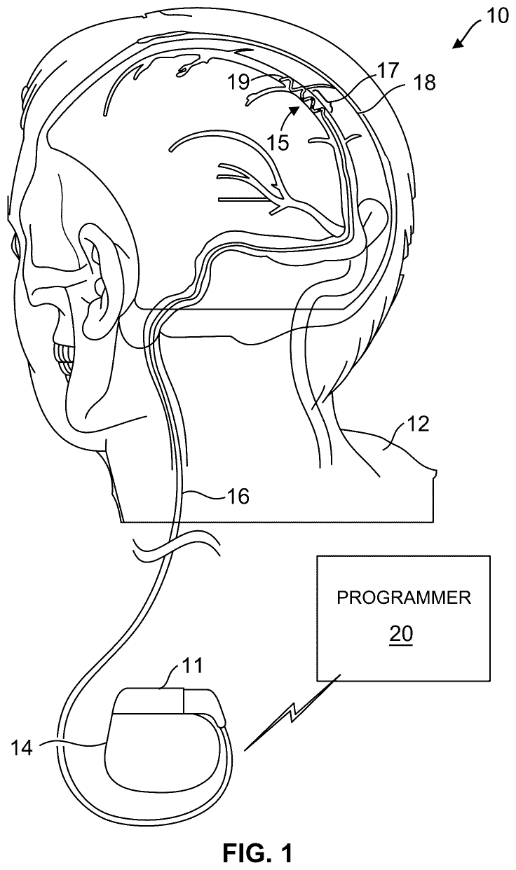

FIG. 1 is a conceptual diagram illustrating an example endovascular therapy system 10 (also referred to herein as therapy system 10 or endovascular system 10) configured to deliver electrical stimulation therapy to a target tissue site in a brain 18 of a patient 12. Additionally and/or alternatively, endovascular therapy system 10 is configured to sense a patient parameter from a target tissue site in a brain 18 of a patient 12. Patient 12 ordinarily will be a human patient. In some cases, however, therapy system 10 may be applied to other mammalian or non-mammalian non-human patients.

In the example of FIG. 1, therapy system 10 includes medical device 14, an endovascular device 16, and a plurality of electrodes 17 disposed on a distal portion 15 of endovascular device 16. In the example shown in FIG. 1, medical device 14 is configured to deliver electrical stimulation therapy to brain 18 of patient 12 and/or sense bioelectrical brain signals from brain 18 via plurality of electrodes 17 of endovascular device 16. Endovascular device 16 is positioned in cranial vasculature of patient 12, such that plurality of electrodes 17 are located proximate to a target tissue site within brain 18 and are positioned to deliver electrical stimulation therapy to brain tissue sites within brain 18 and/or sense one or more patient parameters from the brain tissue sites, such as tissue sites under the dura mater surrounding brain 18. In some examples, placement of endovascular device 16, distal portion 15, and plurality of electrodes 17 is coincident with the dura mater, such as in the middle meningeal artery (MMA). Medical device 14 can provide electrical stimulation to one or more regions within brain 18 in order to manage a condition of patient 12, such as to mitigate the severity or duration of the patient condition, and/or sense one or more patient parameters to provide data and/or feedback required for managing a condition of the patient 12.

Endovascular device 16 includes any elongated body configured to deliver electrical stimulation signals to, and/or sense one or more patient parameters from, tissue proximate plurality of electrodes 17. For example, endovascular device 16 can be and/or include one or more of a medical lead, a catheter, a guidewire, or another elongated body carrying plurality of electrodes 17. One or more portions of endovascular device 16 can be configured to be electrically coupled to medical device 14 either directly or indirectly via one or more electrically conductive pathways (e.g., conductor wires) that runs between medical device 14 and plurality of electrodes 17.

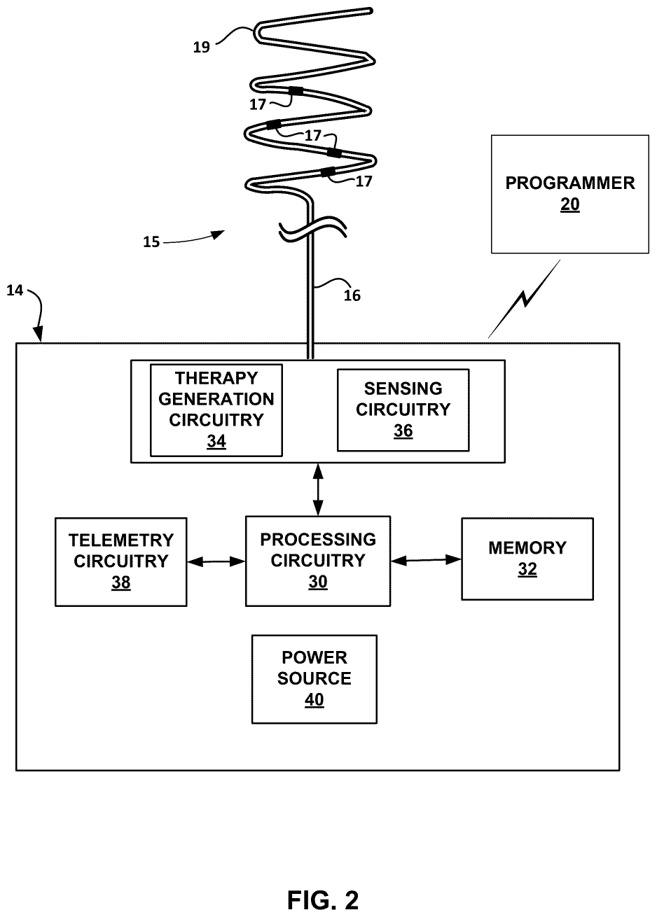

The elongated body of endovascular device 16 is configured to be introduced into a blood vessel of patient 12. Endovascular device 16 has any suitable length that enables connection to medical device 14 either directly or indirectly, e.g., a length of 150 centimeters (cm) to 250 cm, such as 200 cm. As another example, endovascular device 16 can be a wireless therapy delivery device, such as a microstimulator or the like, which is not electrically coupled to medical device 14 via a wired connection. In some of these wireless therapy delivery device examples, system 10 does not include medical device 14 and endovascular device 16 includes therapy generation circuitry and/or other elements of medical device 14 described herein, e.g., with respect to FIG. 2. In some of these wireless therapy delivery device examples, distal portion 15 including plurality of electrodes 17 is configured to detach (e.g., via a detachment mechanism) from an elongated delivery member (e.g., a push wire or a hypotube) used to deliver distal portion 15 to a target site.

In some examples, more than one endovascular device 16 may be positioned within brain 18 of patient 12 to provide stimulation to, and/or sense one or more patient parameters from, multiple anatomical regions of brain 18. Endovascular device 16 can be implanted in a blood vessel for chronic therapy delivery (e.g., on the order of months or even years) or for more temporary therapy delivery (e.g., on the order of days, such as less than a month or less than 6 months). In some examples, one or more devices (e.g., one or more of endovascular device 16) are placed to provide stimulation and/or sense in corresponding regions of the brain, such as in the cortex. In some examples, one or more endovascular devices are placed within intracranial venous structures to provide electrical stimulation and/or sense in corresponding regions of the brain. When endovascular devices are placed in different regions of the brain, for example within multiple arterial locations, multiple venous locations, or within arterial and venous locations (e.g., MMA and deep venous system), the combined sensing from both modalities may provide temporal and spatial data. The temporal and spatial data can be used to control delivery of electrical stimulation therapy to patient 12 and/or to evaluate a patient condition at one point in time or over a longer time period.

Electrical stimulation therapy (e.g., DBS) may be used to treat various patient conditions, such as, but not limited to, seizure disorders (e.g., epilepsy), pain, migraine headaches, psychiatric disorders (e.g., obsessive compulsive disorder, mood disorders or anxiety disorders), movement disorders (e.g., essential tremor or Parkinson's disease), Huntington's disease, and other neurodegenerative disorders. The anatomic region within brain 18 of patient 12 that serve as the target tissue site for electrical stimulation delivered by medical device 14 may be selected based on the patient condition. For example, stimulating an anatomical region, such as the substantia nigra, in brain 18 may reduce the number and magnitude of tremors experienced by patient 12. Other example target anatomical regions for treatment of movement disorders may include the subthalamic nucleus, globus pallidus interna, ventral intermediate, and zona inserta. Anatomical regions such as these may be targeted by the clinician during implantation of endovascular device 16. In other words, the clinician may attempt to position endovascular device 16 within or proximate to these target regions within brain 18 by positioning endovascular device 16 in a cranial blood vessel that is within or proximate to these target regions.

In various examples described herein, example regions of brain 18 that can include the target tissue site for electrical stimulation or sensing via endovascular device 16 positioned in a blood vessel in brain 18 include, but are not limited to, one or more of the anterior thalamus, the ventrolateral thalamus, the subthalamic nucleus (STN), the substantia nigra pars reticulata, the internal segment and/or external segments of the globus pallidus, the ventral intermediate, the zona inserta, the hippocampus (HIP), the dentate gyrus, the cortex (e.g., the motor strip, the sensor strip, the premotor cortex), the fornix, the neostriatum, the ventral intermediate nucleus of the thalamus, the cingulate, or the cingulate gyrus.

The vasculature into which endovascular device 16 may be inserted and/or guided includes, but is not limited to, veins or arteries. For example, to reach certain deep brain tissue sites, endovascular device 16 can be navigated from a vasculature access site (e.g., in the femoral artery, the radial artery, femoral vein, subclavian vein, internal jugular vein or another suitable access site) to one or more arterial structures (including, but not limited to the MMA) or veins of the superficial and a deep venous system (including, but not limited to the thalamostriate vein, the internal cerebral vein, the basal vein of Rosenthal, the inferior sagittal sinus, the superior sagittal sinus, the anterior choroidal artery, or any related combinations thereof).

Certain intracranial blood vessels into which endovascular device 16 may be inserted and/or guided may be located at different distances from different target tissue sites. Such distances may play a role in efficacy of therapy delivered by endovascular device 16, as a closer distance may indicate a shorter distance any electrical stimulation signal may have to travel, and, in some examples, the less power that is needed to generate an efficacious electrical stimulation signal. For example, the thalamostriate vein may be approximately 1.2 millimeters (mm) in diameter and be located approximately 0-2 mm from the anterior nucleus of the thalamus (ANT) and 0-2 mm from the fornix. As another example, the internal cerebral vein may be 1.9 mm plus or minus up to 0.5 mm in diameter and be located approximately 5-10 mm from the ANT and approximately 2-5 mm from the fornix. The basal vein of Rosenthal may be 1.7 mm plus or minus up to 0.4 mm in diameter and be located approximately 10-15 mm from the ANT, approximately 5-10 mm from the HIP, and approximately 5-10 mm from the STN. The inferior sagittal sinus may be 1.3 mm plus or minus up to 0.3 mm in diameter and be located approximately 10-15 mm from the Fornix.

A clinician can also select a particular intracranial blood vessel to position plurality of electrodes 17 at different orientations or distances relative to tissue sites (along with selectively activating groups of electrodes that face a certain direction) for which it may be desirable to avoid electrical stimulation to minimize or even eliminate adverse effects. Electrical stimulation therapy (e.g., DBS) may cause one or more side effects by inadvertently providing electrical stimulation to anatomical regions near the targeted anatomical region. For this reason, a clinician may position plurality of electrodes 17 within brain 18 and/or program the electrical stimulation parameters in order to balance effective therapy and minimal side effects.

As discussed in further detail below, in some examples, endovascular device 16 is configured to be delivered to one or more target sites in brain 18 via vasculature of patient 12. Thus, rather than introducing endovascular device 16 into brain tissue (e.g., the cerebral parenchyma) via a burr hole through a skull of patient 12 or the like, endovascular device 16 is configured to be navigated to a target electrical stimulation site in brain 18 via vasculature of patient 12. The endovascular delivery of endovascular device 16 to deep brain sites in brain 18 can help minimize the invasiveness of therapy system 10.

Endovascular device 16 may include an elongated body that is structurally configured to be relatively flexible, pushable, and relatively kink-and buckle-resistant, so that it may resist buckling when a pushing force is applied to a relatively proximal portion to advance endovascular device 16 distally through vasculature, and so that it may resist kinking when traversing around a tight turn in the vasculature. Kinking and/or buckling of may hinder a clinician's efforts to push the elongated body distally, e.g., past a turn. In some examples, endovascular device 16 includes one or more radiopaque components (e.g., platinum bands and/or other structures including platinum or platinum alloys) proximate electrodes 17.

In some examples, endovascular device 16 can be navigated through vasculature (e.g., to brain 18 or other target tissue sites) with the aid of a guide member. The guide member can include an outer catheter, an inner catheter, a guide extension catheter, a guidewire, or the like or combination thereof.

In some examples, as discussed herein, plurality of electrodes 17 are positioned on a portion of endovascular device 16 that may be configured to deploy (e.g., expand) radially outwards from a relatively low-profile delivery configuration to a deployed (e.g., coiled and/or expanded) configuration. For example, as illustrated in the example of FIG. 1, endovascular device includes a coil structure 19 positioned at distal portion 15 of endovascular device 16. Electrodes 17 may be positioned on a portion of (e.g., a proximal portion of) coil structure 19.

In some examples, at least a portion of coil structure 19 is configured to hold electrodes 17 in apposition with a blood vessel wall. Holding electrodes 17 in apposition with a blood vessel wall may promote more efficient therapy delivery (e.g., due to less electrical power needed to transmit therapeutically sufficient electrical stimulation). Additionally and/or alternatively, holding electrodes 17 in apposition with a blood vessel wall promote tissue ingrowth or endothelization around plurality of electrodes 17 along the vessel wall (while still maintaining a vessel lumen).

In some examples, as discussed herein, coil structure 19 is configured to anchor the endovascular device 16, within the vasculature of patient 12. For example, coil structure 19 can be introduced into a blood vessel and configured to exert a radial force against a blood vessel wall (e.g., radially outward from a radial center of the blood vessel). Such force may both urge electrodes 17 towards a wall of the blood vessel as well as exert a radial force sufficient to anchor endovascular device 16, including coil structure 19, within the vasculature of patient 12.

In some examples, coil structure 19 is configured to transform between a relatively low-profile delivery configuration and a deployed configuration. In the relatively low-profile delivery configuration, coil structure 19 may be configured to be collapsed to a smaller profile (e.g., having a smaller maximum radial dimension) and loaded into a delivery device, which may be navigated to a target location with the vasculature. In the relatively low-profile delivery configuration, coil structure 19 can be straight or substantially straight (e.g., to a level sufficient to reside within a lumen of a delivery catheter and/or sheath). In the deployed configured, coil structure 19 may be configured to radially expand outward, such as to provide a radially outward force (e.g., to urge the elongated body of endovascular device 16 and plurality of electrodes 17 towards a wall of the blood vessel). When coil structure 19 is in the deployed configuration, electrodes 17 may be in a position to deliver electrical stimulation to tissue of the patient 12 or sense a patient parameter (e.g., a signal, such as a bioelectric signal) from a location within the vasculature of patient 12.

In some examples, coil structure 19 can include a pre-shaped coil (e.g., a pre-shaped metal coil). For example, coil structure 19 can be configured to assume a predetermined coil shape in the absence of external forces. Coil structure 19 can include one or more materials that enable coil structure 19 to define the predetermined coil shape (e.g., Pt-W). In some examples, coil structure 19 is self-expanding or at least partially self-expanding.

In some examples, coil structure 19 is expanded (or at least partially expanded) via a suitable expansion mechanism (e.g., by a balloon, via a pullwire, via electrical energy, via thermal energy, etc.). As described herein, coil structure 19 may include a suitable structure, material, or combination thereof to exert a radially force outward (e.g., toward a blood vessel wall).

In some examples, at least distal portion 15 (e.g., coil structure 19) of endovascular device 16 includes a shape memory material (e.g., nitinol) material that enables distal portion 15 to assume a predetermined shape in the absence of a force (e.g., a compressive or tensile force) holding the distal portion 15 in a relatively low-profile delivery configuration. For example, at least coil structure 19 of endovascular device 16 can include the shape memory material.

Coil structure 19 of endovascular device 16 can be configured to expand radially outwards upon deployment from an outer sheath (e.g., an outer catheter), or upon the proximal withdrawal of a straightening element (e.g., a guidewire or a mandrel) positioned in an inner lumen of endovascular device 16. As another example, the distal portion 15 can be configured to expand radially outwards in response to proximal withdrawal of a pull member attached to distal portion 15 of the endovascular device 16 or in response to a distal movement of an elongated control member attached to distal portion 15.

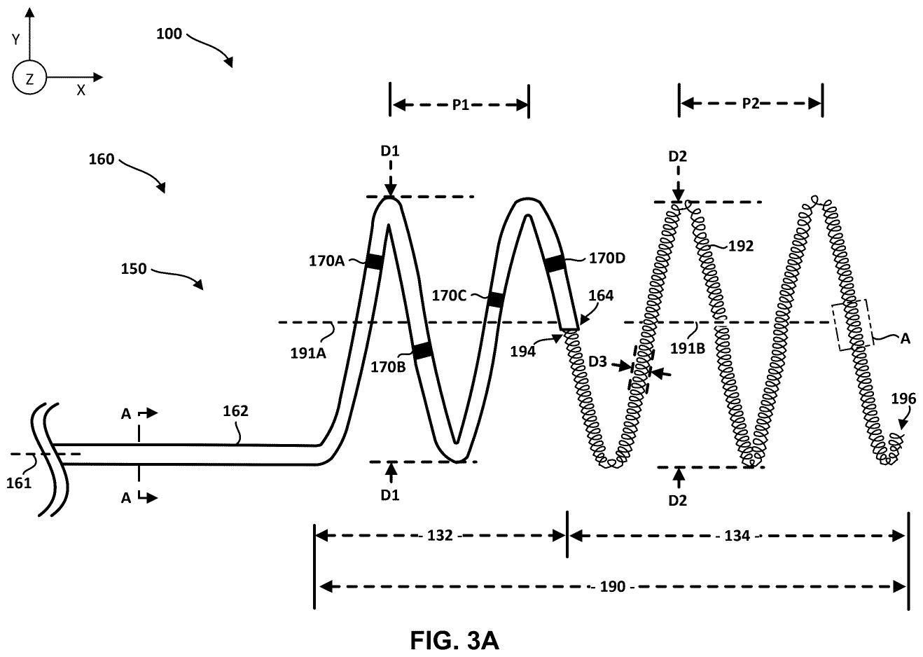

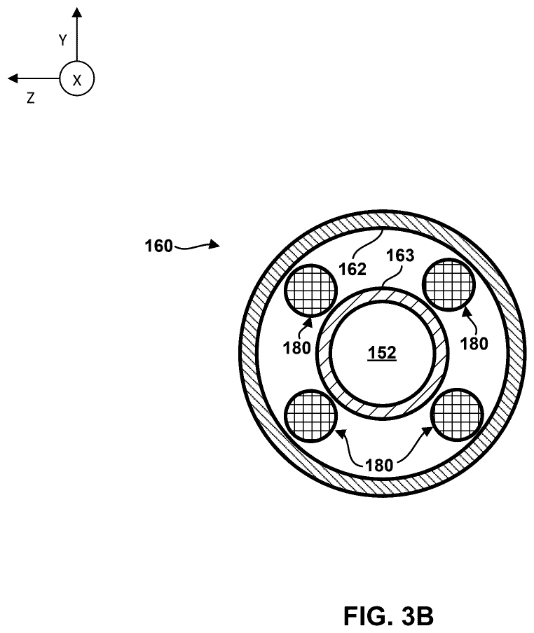

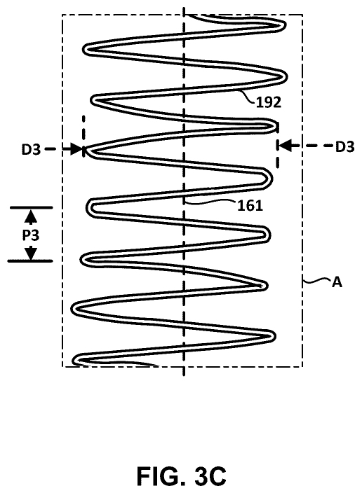

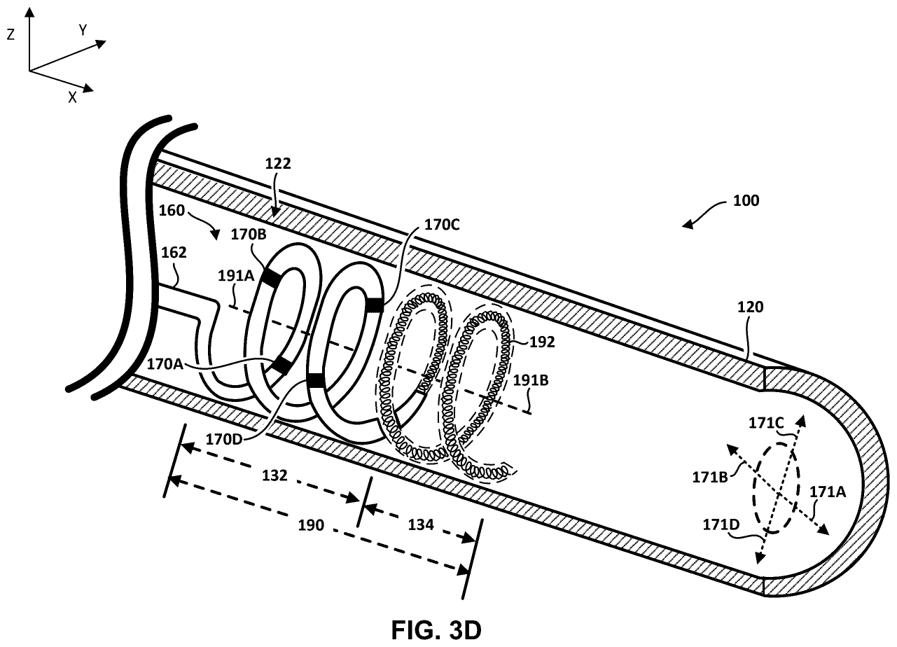

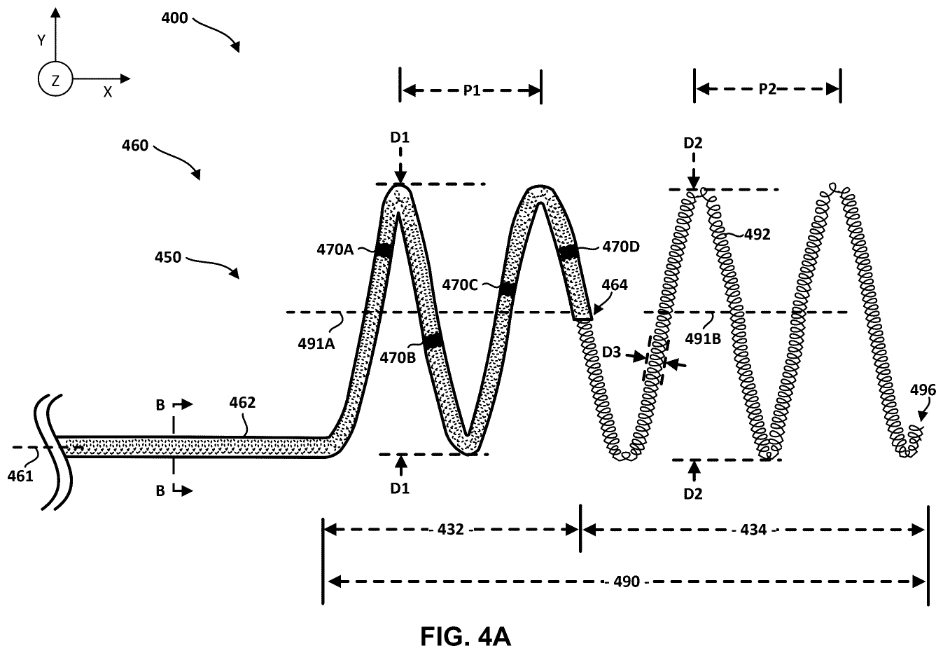

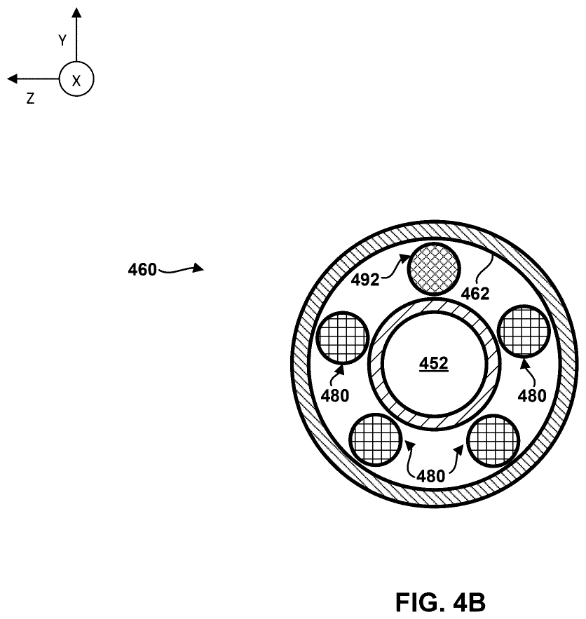

In some examples, as discussed further with respect to at least FIG. 3A and FIG. 4A, coil structure 19 can include multiple portions having different configurations (e.g., different shape, form factors, materials, and/or other characteristics). In some examples, coil structure 19 of endovascular device 16 includes at least a first coil portion that carries electrodes 17 and a second coil portion configured to help anchor endovascular device 16, including the first coil portion, within the vasculature of the patient 12. The first coil portion of coil structure 19 can be a coil shape defined by a portion of the elongated body of endovascular device 16. In some examples, the second coil portion of coil structure 19 can include and/or define one or more of a different shape, form factor, material, and/or other characteristics as compared to the first coil portion. For example, as discussed in relation to at least FIG. 3A, the second coil portion of coil structure 19 includes a coiled wire mechanically coupled to the first coil portion of the coil structure 19. In some examples, the first coil portion of coil structure 19 is a proximal coil portion and the second coil portion of coil structure 19 is a distal coil portion.

In some examples, as discussed in relation to at least FIG. 4A, the second coil portion includes a coiled wire that is at least partially coextensive with the elongated body of endovascular device 16 (e.g., coextensive with one or more conductor wires that extend within the endovascular device 16). The second coil portion of coil structure 19 can be configured to provide additional and/or complimentary anchoring as compared to the first coil portion of coil structure 19.

In some examples, the first coil portion and the second coil portion of coil structure 19 define a continuous or substantially continuous coil shape (e.g., a continuous coil shape at distal portion 15 of endovascular device 16). For example, the first coil portion of coil structure 19 can include a diameter and pitch (e.g., spacing) that is the same or substantially the same as a diameter and pitch of the second coil portion of coil structure 19. The continuous and/or the substantially continuous coil shape formed by the first coil portion and the second coil portion of coil structure 19 can be relatively easier to transform to a relatively low-profile delivery configuration, which can facilitate easier delivery and/or retrieval of endovascular device 16 and/or coil structure 19. The relatively simple, continuous shape of the coil formed by the first coil portion and the second coil portion of coil structure 19 (e.g., as compared to other expandable anchoring structures, such as stents and/or stent-like structures) can enable relatively simpler or easier manufacturing and/or assembly.

In one or more examples, the second coil portion of coil structure 19 includes a coiled wire. For example, the second coil portion of coil structure 19 can include a coiled wire such that the second coil portion is a coiled coil. Using a coiled wire that forms a coil (e.g., such as to define a coiled coil) for anchoring the endovascular device 16 and electrodes 17 within the vasculature of patient 12 can facilitate relatively stronger anchoring, e.g., as compared to having a coil portion the main body portion of endovascular device 16, which can have a relatively smooth outer surface. For example, a coiled wire may facilitate relatively greater engagement (e.g., contact, friction, and/or the like) with a vessel wall causing the endovascular device 16 to be relatively more firmly anchored within vasculature of the patient as compared to other anchoring mechanisms.

Electrodes 17 can have any suitable configuration and arrangement, including a full ring, segmented, and/or partial ring configuration. In some examples, some or all of plurality of electrodes 17 are integrally formed with at least distal portion 15 of endovascular device 16. For example, at least distal portion 15 of endovascular device 16 is formed from an electrically conductive material that is electrically connected to therapy generation circuitry 34 and sensing circuitry 36 (FIG. 2) and an electrically insulative material can be positioned radially outwards of the electrically conductive material to cover the electrically conductive material. In such examples, where endovascular device 16 includes an electrically insulative material, at least a portion of the electrically insulative material is removed to expose the electrically conductive material to define plurality of electrodes 17. To define plurality of electrodes 17, part of the electrically insulative material can be removed (e.g., via laser ablation, mechanical etching, or the like) to expose the electrically conductive material. Any suitable electrically insulative material can be used, such as, but not limited to, polytetrafluoroethylene (PTFE), expanded PTFE (ePTFE), silicone, polyimide, non-metallic oxide, parylene or the like. The electrically insulative material can have any suitable thickness, such as, but not limited to, 0.010 mm to 0.05 mm (e.g., about 0.0005 inches).

In other examples, plurality of electrodes 17 are or include a component physically separate from endovascular device 16 and mechanically connected to the endovascular device 16. For example, plurality of electrodes 17 includes an electrically conductive electrode material (e.g., platinum, tungsten, gold, or the like, which can be radiopaque or not) electrically coupled to one or more electrically conduct materials extending through endovascular device 16.

Plurality of electrodes 17 and/or endovascular device 16 (e.g., including coil structure 19) can include one or more surface textures or coatings to promote endothelization, decrease impedance, reduce thrombosis, or increase longevity. Materials for such structures and/or coatings may include one or more of Titanium Nitride, Platinum (e.g., with laser texturing and/or with iridium oxide (IrOx)), and/or or poly(3,4-ethylenedioxythiophene) polystyrene sulfonate (“PEDOT:PSS” or “PDOT”). The one or more surface textures or coatings may increase a surface area of plurality of electrodes 17, which can help stabilize the impedance over a range of frequencies (e.g., of an electrical stimulation signal).

Medical device 14 can be an external medical device or an implantable medical device that includes electrical stimulation circuitry configured to generate and deliver electrical stimulation therapy to patient 12 via plurality of electrodes 17 of endovascular device 16. Plurality of electrodes 17 may be configured to deliver electrical stimulation to tissue of brain 18 of patient 12 from a location within a blood vessel.

In the example of FIG. 1, endovascular device 16 is directly or indirectly mechanically and electrically coupled to medical device 14 via a header 11 of medical device 14. In some examples, header 11 defines a plurality of electrical contacts in one or more feedthrough portions (e.g., that are configured to electrically couple electrodes 17 to electrical stimulation generation circuitry and/or sensing circuitry within medical device 14).

In some examples, endovascular therapy system 10 includes one or more conductor wires (not shown in FIG. 1) extending between medical device 14 and electrodes 17. The one or more conductor wires can be configured to carry electrical signals between medical device 14 and electrodes 17 or vice versa. The conductor wires may extend along, be a part of, be incorporated into, and/or integrally formed as part of endovascular device 16. Other types of conductive elements for transmitting electrical signals (e.g., trace elements and the like) can additionally or alternatively be used. In some examples, header 11 includes multiple feedthrough portions, which may be respectively configured for receiving one of multiple portions of endovascular device 16. Header 11 may also be referred to as a connector block or connector of medical device 14. Endovascular device 16 may be mechanically coupled and/or electrically coupled to header 11 with the aid of a lead extension. However, in some examples, a lead extension is not used between header 11 and endovascular device 16, and endovascular device is directly mechanically and/or electrically connected to medical device 14 via header 11.

In some examples, medical device 14 is configured to be implanted in patient 12 in any suitable location, such as a location outside of brain 18, e.g., in a pectoral region. In other examples, medical device 14 is configured to be external to patient 12. Endovascular device 16 may be, for example, implanted within a cranial vein and one or more proximal wires/leads can remain within the venous system until they exit the subclavian vein in the chest for implant in the pectoral region. In yet other examples, some or all of medical device 14 is configured to be implanted in brain 18, e.g., as part of endovascular device 16.

As shown in FIG. 1, system 10 may also include a programmer 20, which may be a handheld device, portable computer, or workstation that provides a user interface to a clinician or other user. The clinician may interact with the user interface to program electrical stimulation parameters for medical device 14.

With the aid of programmer 20 or another computing device, a clinician may select values for therapy parameters for controlling therapy delivery by therapy system 10. The values for the therapy parameters may be organized into a group of parameter values referred to as a “therapy program” or “therapy parameter set.” “Therapy program” and “therapy parameter set” are used interchangeably herein. In the case of electrical stimulation, the therapy parameters may include an electrode combination, a power, and an amplitude, which may be a current or voltage amplitude, and, if medical device 14 delivers electrical pulses, a pulse width, and a pulse rate or frequency for stimulation signals to be delivered to the patient. Other example therapy parameters include a slew rate, duty cycle, and phase of the electrical stimulation signal. An electrode combination may include a selected group (e.g., electrodes that face the same direction) or subset (e.g., less than all of the electrodes) of plurality of electrodes 17 located on one or more implantable elongated bodies (such as endovascular device 16) coupled to medical device 14. The electrode combination may also refer to the polarities of the electrodes in the selected subset. By selecting particular electrode combinations, a clinician may target particular anatomic structures within brain 18 of patient 12. In addition, by selecting values for slew rate, duty cycle, phase amplitude, pulse width, and/or pulse rate, the clinician can attempt to generate an efficacious therapy for patient 12 that is delivered via the selected electrode subset.

Whether programmer 20 is configured for clinician or patient use, programmer 20 may communicate with medical device 14 or any other computing device via wireless or a wired communication. Programmer 20, for example, may communicate via wireless communication with medical device 14 using radio frequency (RF) telemetry techniques known in the art. Programmer 20 may also communicate with another programmer or computing device via a wired or wireless connection using any of a variety of local wireless communication techniques, such as RF communication according to the 802.11 or Bluetooth specification sets, infrared communication according to the Infrared Data Association (IRDA) specification set, or other standard or proprietary telemetry protocols. Programmer 20 may also communicate with another programming or computing device via a wired or wireless communication technique.

In some examples, in addition to or instead of delivering electrical stimulation to brain 18, endovascular device 16 can be used to sense one or more patient parameters, such as bioelectrical signals, either using plurality of electrodes 17 or other types of sensors that are carried by endovascular device 16. In some examples, a sensed patient parameter includes an impedance detected via plurality of electrodes 17. In some examples, the bioelectrical signals sensed within brain 18 reflect changes in electrical current produced by the sum of electrical potential differences across brain tissue. Examples of bioelectrical brain signals that can be sensed via one or more electrodes of plurality of electrodes 17 include, but are not limited to, electrical signals generated from local field potentials within one or more regions of brain 18, an electroencephalogram (EEG) signal, an electrocorticogram (ECoG) signal, or an evoked potential. In some examples, a sensing parameter includes one or more of a direction faced by each sensing electrode or group of electrodes (e.g., the active electrodes with which a medical device senses a patient parameter), a location of one or more electrodes within brain 18, or other parameters that may affect detection and/or sensing of one or more patient parameters.

Brain 18 in FIG. 1 is supplied with blood through the carotid and the vertebral arteries on each side of the neck. The arteries include the common carotid artery in the neck, which is a common access pathway for the various devices and/or methods disclosed herein, the internal carotid which supplies the ophthalmic artery. The external carotid supplies the maxillary artery, the middle meningeal artery (MMA), and the superficial temporal arteries (frontal and parietal). The vertebral artery supplies the basilar artery and the cerebral arteries including the posterior cerebral artery and the circle of Willis. The siphon of the vertebral artery appears in the intra-cranial vasculature on the vertebral approach to the Circle of Willis. Also supplied by the internal carotid artery are the anterior cerebral artery and the middle cerebral artery (MCA), as well as the circle of Willis, including the posterior communicating artery and the anterior communicating artery. The siphon of the internal carotid artery appears in the intra-cranial vasculature on the carotid approach into the Circle of Willis. These arteries can have an internal diameter of about 1 mm to 5 mm, most commonly from 2-4 mm.

The devices, systems, and methods described herein enable endovascular delivery to deep brain tissue sites in brain 18. Endovascular device 16 can be navigated to the cranial vasculature to reach the deep brain tissue sites, e.g., via an insertion catheter (e.g., a microcatheter). As an example, endovascular device 16 can be delivered to an intracranial blood vessel inside of a 0.017 inch (about 0.43 mm) or a 0.021 inch (about 0.53 mm) microcatheter, alone or with the aid of a guidewire.

In addition to, or instead of, chronic therapy delivery and/or chronic sensing, example devices, systems, and methods described herein can be used for more temporary applications. In some examples, a first endovascular device (e.g., configured like endovascular device 16 or having another configuration) is configured to be operated in an acute (e.g., temporary) trial mode for a trial period to determine, evaluate, or confirm an efficacy of stimulation and/or sensing. For example, endovascular device 16 may be configured to operate in the trial mode to determine the efficacy of one or more stimulation parameter values and/or one or more sensing parameters. After the acute trial period, the first endovascular device may be removed, and a second endovascular device (e.g., configured like endovascular device 16 or having another configuration) configured to operate in a chronic mode may be implanted for a chronic period for chronic (e.g., long term, or permanent) stimulation therapy or sensing. In some examples, a first endovascular device (e.g., for use in the acute trial mode) is configured to be implanted and subsequently removed after the trial period.

FIG. 2 is a functional block diagram illustrating components of an example medical device 14, which is configured to generate and deliver electrical stimulation therapy to patient 12 and, in some examples, sense one or more patient parameters, such as bioelectrical brain signals of patient 12. Medical device 14 includes processing circuitry 30, memory 32, therapy generation circuitry 34, sensing circuitry 36, telemetry circuitry 38, and power source 40.

Therapy generation circuitry 34 includes any suitable configuration (e.g., hardware) configured to generate electrical stimulation signals to a target tissue site in brain 18 of patient 12. Processing circuitry 30 is configured to control therapy generation circuitry 34 to generate and deliver electrical stimulation therapy via plurality of electrodes 17 of endovascular device 16. Plurality of electrodes 17 may include a monopolar or bipolar arrangement. The electrical stimulation parameter values may be selected based on the patient condition being addressed, as well as the target tissue site in brain 18 for the electrical stimulation therapy. The electrical stimulation therapy can be provided via stimulation signals of any suitable form, such as stimulation pulses or continuous-time signals (e.g., sine waves).

Sensing circuitry 36 is configured to sense a physiological parameter of a patient. Sensing circuitry 36 may include any sensing hardware configured to sense a physiological parameter of a patient, such as, but not limited to, one or more electrodes, optical receivers, pressure sensors, or the like. The one or more sensing electrodes can be the same or different from plurality of electrodes 17 configured to deliver electrical stimulation therapy. Processing circuitry 30 can use the sensed physiological signals to control therapy delivery by therapy generation circuitry 34, e.g., the timing of the therapy delivery or one or more characteristics of the electrical simulation signal generated by therapy generation circuitry 34.

In some examples, sensing circuitry 36 is configured to sense a bioelectrical brain signal via plurality of electrodes 17 (e.g., all or a subset of electrodes 17). Thus, plurality of electrodes 17 can be configured to receive or transmit energy (e.g., current). Example bioelectrical brain signals include an EEG signal, an ECoG signal, a signal generated from measured field potentials within one or more regions of brain 18, action potentials from single cells within brain 18 (referred to as “spikes”), or evoked potentials. Determining action potentials of single cells within brain 18 may require resolution of bioelectrical signals to the cellular level and provides fidelity for fine movements, i.e., a bioelectrical signal indicative of fine movements (e.g., slight movement of a finger). In examples in which endovascular device 16 is configured to sense an evoked potential, endovascular device 16 may also be configured to generate a stimulus (e.g., via therapy generation circuitry, alone or in combination with processing circuitry 30) to elicit the evoked potential. For example, endovascular device 16 can generate and deliver electrical stimulation to tissue in brain 18 and sense an evoked compound action potential (ECAP). An ECAP is synchronous firing of a population of neurons which occurs in response to the application of a stimulus including, in some cases, an electrical stimulus by endovascular device 16. The ECAP may be detectable as being a separate event from the stimulus itself, and the ECAP may reveal characteristics of the effect of the stimulus on the tissue.

In some examples, sensing circuitry 36 and/or processing circuitry 30 includes signal processing circuitry configured to perform any suitable analog conditioning of the sensed physiological signals. For example, sensing circuitry 36 may communicate to processing circuitry 30 an unaltered (e.g., raw) signal. Processing circuitry 30 may be configured to modify a raw signal to a usable signal by, for example, filtering (e.g., low pass, high pass, band pass, notch, or any other suitable filtering), amplifying, performing an operation on the received signal (e.g., taking a derivative, averaging), performing any other suitable signal conditioning (e.g., converting a current signal to a voltage signal), or any combination thereof. In some examples, the conditioned analog signals may be processed by an analog-to-digital converter of processing circuitry 30 or other component to convert the conditioned analog signals into digital signals. In some examples, processing circuitry 30 may operate on the analog or digital form of the signals to separate out different components of the signals. In some examples, sensing circuitry 36 and/or processing circuitry 30 may perform any suitable digital conditioning of the converted digital signals, such as low pass, high pass, band pass, notch, averaging, or any other suitable filtering, amplifying, performing an operation on the signal, performing any other suitable digital conditioning, or any combination thereof. Additionally or alternatively, sensing circuitry 36 may include signal processing circuitry to modify one or more raw signals and communicate to processing circuitry 30 one or more modified signals.

Although shown as part of medical device 14 in FIG. 2, in other examples, sensing circuitry 36 can be a part of a device separate from medical device 14. For example, sensing circuitry 36 can be part of an implantable sensing device implanted in cranial vasculature or elsewhere in brain 18 of patient 12.

Processing circuitry 30, as well as other processors, processing circuitry, controllers, control circuitry, and the like, described herein, may include any combination of integrated circuitry, discrete logic circuity, analog circuitry, such as one or more microprocessors, digital signal processors (DSPs), application specific integrated circuits (ASICs), or field-programmable gate arrays (FPGAs). In some examples, control circuitry may include multiple components, such as any combination of one or more microprocessors, one or more DSPs, one or more ASICs, or one or more FPGAs, as well as other discrete or integrated logic circuitry, and/or analog circuitry.

Memory 32 is configured to store program instructions, such as software, which may include one or more program modules, which are executable by processing circuitry 30. When executed by processing circuitry 30, such program instructions may cause processing circuitry 30 to provide the functionality ascribed to processing circuitry 30 herein. The program instructions may be embodied in software and/or firmware. Memory 32 may include any volatile, non-volatile, magnetic, optical, or electrical media, such as a random-access memory (RAM), read-only memory (ROM), non-volatile RAM (NVRAM), electrically-erasable programmable ROM (EEPROM), flash memory, or any other digital media.

Processing circuitry 30 is configured to control telemetry circuitry 38 to send and receive information. Telemetry circuitry 38, as well as telemetry modules in other devices described herein, such as programmer 20, may accomplish communication by any suitable communication techniques, such as RF communication techniques. In addition, telemetry circuitry 38 may communicate with programmer 20 via proximal inductive interaction of medical device 14 with programmer 20. Accordingly, telemetry circuitry 38 may send information to programmer 20 on a continuous basis, at periodic intervals, or upon request from medical device 14 or programmer 20.

Power source 40 is configured to deliver operating power to various components of medical device 14. Power source 40 may include a small rechargeable or non-rechargeable battery and a power generation circuit to produce the operating power. Recharging may be accomplished through proximal inductive interaction between an external charger and an inductive charging coil within medical device 14. In some examples, power requirements may be small enough to allow medical device 14 to utilize patient motion and implement a kinetic energy-scavenging device to trickle charge a rechargeable battery. In other examples, traditional batteries may be used for a limited period of time.

As discussed above, in some examples, endovascular device 16 is configured to be a standalone electrical stimulation device and can include one or more elements of medical device 14 shown in FIG. 2.

FIG. 3A illustrates an example endovascular therapy system 100, which may be an example of therapy system 10 of FIG. 1. FIG. 3A illustrates a side view of endovascular therapy system 100. Endovascular therapy system 100 includes endovascular device 160 including a coil structure 190 at a distal portion 150 of endovascular device 160. Endovascular device 160 can be and/or include a medical lead. As illustrated, endovascular therapy system 100 includes a plurality of electrodes 170 (shown individually as electrode 170A, electrode 170B, electrode 170C, and electrode 170D, but collectively referred to herein as plurality of electrodes 170) at distal portion 150 of endovascular device 160. Endovascular device 160, distal portion 150, coil structure 190, and electrodes 170 are examples of endovascular device 16, distal portion 15, coil structure 19, and electrodes 17 as illustrated in and described with respect to FIG. 1, respectively.

FIG. 3B illustrates a cross-sectional view of a portion of endovascular therapy system 100 of FIG. 3A. In the example of FIG. 3B, the cross-section is taken through the A-A section lines of FIG. 3A and faces in the positive x-axis direction according to the orthogonal x-y-z axes of FIG. 3A. FIG. 3C illustrates a detail view of a portion of endovascular therapy system 100 illustrated in FIG. 3A, the detail view including the portion of endovascular therapy system 100 enclosed by the dashed line box indicated as “A” in the example of FIG. 3A.

FIG. 3D illustrates endovascular therapy system 100 of FIG. 3A positioned within a blood vessel 120 having a blood vessel wall 122. Blood vessel 120 may be an example of a suitable neurovascular blood vessel, a jugular vein, and/or another suitable blood vessel into which endovascular therapy system 100 can be positioned. As illustrated in the example of FIG. 3D, coil structure 190 is in the deployed configuration such that electrodes 170 are in apposition with and/or contacting blood vessel wall 122.

Endovascular device 160 can have any suitable configuration, and may be configured according to the description of endovascular device 16 of FIG. 1. In some examples, and with reference to at least FIG. 3A and FIG. 3B, endovascular device 160 includes an elongated body 162. Elongated body 162 can be a tubular body defining at least one lumen (e.g., a lumen 152 as illustrated in the example of FIG. 3B). In some examples, endovascular device 160 includes an inner elongated body 163 (e.g., positioned within elongated body 162, which may be an outer elongated body). In some examples, inner elongated body 163 defines lumen 152.

Coil structure 190 can be positioned at distal portion 150 of elongated body 162. With reference to FIG. 3A, elongated body 162 of endovascular device 160 can extend between an elongated body proximal end (not show in the examples of FIG. 3A) and an elongated body distal end 164. Elongated body distal end 164 may be a distalmost end of elongated body 162.

In some examples, at least a portion (e.g., a distal portion) of elongated body 162 forms at least a portion of coil structure 190. For example, at least a portion of elongated body 162 can define a coil shape (e.g., pre-shaped coil) to form at least a portion of coil structure 190.

In some examples, elongated body 162 of endovascular device 160 defines an elongated body central longitudinal axis 161 extending along endovascular device 160. Elongated body central longitudinal axis 161 may be a central longitudinal axis of one or more of elongated body 162 and/or endovascular device 160.

In some examples, endovascular device 160 (e.g., at least elongated body 162) includes a suitable biocompatible polymer material. For example, elongated body 162 of endovascular device 160 can include a thermoplastic material, such as Polycarbonate Urethane (PCU). In some examples, elongated body 162 of endovascular device 160 can additionally or alternatively include one or more of Polyurethane (PUR or PU), Polyethylene (PE), Polypropylene (PP), Polyetheretherketone (PEEK), Polyphenylsulfone (PPSU or PPSF), Polypropylene (PP), Nylon, Polyester, Polyethylene Terephthalate (PET), Polymethyl Methacrylate (PMMA), Polysulfone (PSU), and/or another suitable material.

As illustrated in the example of at least FIG. 3A and FIG. 3D, electrodes 170 are carried by a portion of coil structure 190. In some examples, coil structure 190 is configured to position and/or orient electrodes 170 within vasculature of a patient (e.g., patient 12 of FIG. 1). In some examples, as illustrated in the example of at least FIG. 3A and FIG. 3D, electrodes 170 are carried by and/or disposed on a proximal portion of coil structure 190.

Coil structure 190 can be configured to anchor endovascular device 160, including elongated body 162 and electrodes 170, within the vasculature of a patient (e.g., within blood vessel 120 of FIG. 3D). For example, coil structure 190 can be to be introduced into blood vessel 120 and configured to exert a radial force against blood vessel wall 122 of blood vessel 120 (e.g., radially outward from a radial center of blood vessel 120). Such force may both urge electrodes 170 towards blood vessel wall 122 as well as exert a radial force sufficient to anchor endovascular device 160, including coil structure 190, within blood vessel 120.

Coil structure 190 may be configured to transform between a relatively low-profile delivery configuration and a deployed configuration (e.g., an expanded configuration, such as the configuration illustrated in at least FIG. 3A and FIG. 3D). In the relatively low-profile delivery configuration, coil structure 190 may be configured to be collapsed to a smaller profile (e.g., having a smaller maximum radial dimension) and loaded into a delivery device, which may be navigated to a target location with the vasculature. In the relatively low-profile delivery configuration, coil structure 190 can be straight or substantially straight (e.g., to a level sufficient to reside within a lumen of a delivery catheter and/or sheath). In the deployed configured, coil structure 190 may be configured to radially expand outward, such as to provide a radially outward force. In some examples, and with reference to FIG. 3D, the radially outward force provided by coil structure 190 urges electrodes 170 towards blood vessel wall 122 of blood vessel 120.

When coil structure 190 is in the deployed configuration, electrodes 170 may be in a position to deliver electrical stimulation to tissue a patient or sense a patient parameter (e.g., a signal, such as a bioelectric signal) from within blood vessel 120. For example, and with reference to FIG. 3D, when electrodes 170 are in apposition and/or in contact with vessel wall 122 of blood vessel 120, a medical device can be configured to delivery electrical stimulation therapy and/or sense a patient parameter from tissue (e.g., brain tissue and/or one or more nerves) surrounding blood vessel 120.

In some examples, coil structure 190 includes multiple portions having different characteristics (e.g., different shape, form factors, materials, and/or other characteristics). In some examples, as illustrated in at least FIG. 3A, coil structure 190 of endovascular device 160 includes at least a first coil portion 132 and a second coil portion 134. Elongated body central longitudinal axis 161 can be a central longitudinal axis of one or more of elongated body 162, first coil portion 132, and/or second coil portion 134.

In the example of FIG. 3A, electrodes 170 are carried by first coil portion 132 of coil structure 190. In some examples, as illustrated in the example of FIG. 3A, second coil portion 134 does not include any of electrodes 170 (e.g., such that all electrodes 170 of endovascular therapy system 100 are carried by first coil portion 132).

First coil portion 132 and second coil portion 134 can include and/or define any suitable shape and/or configuration. In some examples, first coil portion 132 is a continuous extension of elongated body 162 of endovascular device 160. For example, first coil portion 132 can include a coil shape defined by a portion of elongated body 162 of endovascular device 160 (e.g., by a portion of endovascular device 160 proximal of elongated body distal end 164). First coil portion 132 can define a pre-shaped coil shape. For example, first coil portion 132 can include a molded and/or a shape-set (e.g., heat-set) portion of elongated body 162. Elongated body distal end 164 may also be a distal end (e.g., a distalmost end) of first coil portion 132. First coil portion 132 can include one or more common materials with elongated body 162. In some examples, first coil portion 132 includes a polymer (e.g., which may be the same or a similar polymer that forms elongated body 162). In some examples, first coil portion 132 includes one or more additional materials (e.g., as compared to a more proximal portion of elongated body 162), such as Pt-W, nitinol, another polymer, and/or the like.

In the example of FIG. 3A, first coil portion 132 defines a first central longitudinal coil axis 191A. First central longitudinal coil axis 191A can extend through a radial center of first coil portion 132 (e.g., in the positive and negative x-axis directions according to the orthogonal x-y-z axes of FIG. 3A). First coil portion 132 (e.g., the coil loops of first coil portion 132) can extend around first central longitudinal coil axis 191A. In some examples, first coil portion 132 of coil structure 190 is configured to transform (e.g., expand) from a delivery (e.g., relatively low-profile) configuration to a deployed (e.g., expanded) configuration relative to first central longitudinal coil axis 191A.

In some examples, second coil portion 134 is a physically unique (e.g., isolated) component as compared to first coil portion 132. Second coil portion 134 can be mechanically coupled to (e.g., fixedly mechanically coupled to) first coil portion 132. In the example of FIG. 3A, second coil portion 134 extends between a second coil portion proximal end 194 and a second coil portion distal end 196 (e.g., which may be a distalmost end of second coil portion 134). In the example of FIG. 3A, second coil portion 134 defines a second central longitudinal coil axis 191B. Second central longitudinal coil axis 191B can extend through a radial center of second coil portion 134 (e.g., in the positive and negative x-axis directions according to the orthogonal x-y-z axes of FIG. 3A). Second coil portion 134 (e.g., the coil loops of second coil portion 134) can extend around second central longitudinal coil axis 191B. In some examples, second coil portion 134 of coil structure 190 is configured to transform (e.g., expand) from a delivery (e.g., relatively low-profile) configuration to a deployed (e.g., expanded) configuration relative to second central longitudinal coil axis 191B. The configuration of second coil portion 134 as illustrated in FIG. 3A and/or FIG. 3D can be an example of the deployed (e.g., expanded) configuration of second coil portion 134.

While first central longitudinal coil axis 191A and second central longitudinal coil axis 191B and shown as separate axes, first central longitudinal coil axis 191A and second central longitudinal coil axis 191B can be coaxial such that first central longitudinal coil axis 191A and second central longitudinal coil axis 191B form a common axis (e.g., which can be a common axis of coil structure 190). In examples in which first central longitudinal coil axis 191A and second central longitudinal coil axis 191B form a common axis, the common axis is referred to as central coil longitudinal axis. In other examples, first central longitudinal coil axis 191A and second central longitudinal coil axis 191B are not coaxial (e.g., are offset in one or more of the y-axis and z-axis directions according to the orthogonal x-y-z axes of FIG. 3A).

In some examples, coil structure 190 is configured to expand (e.g., self-expand and/or via an expansion mechanism such as a balloon) radially outward relative to one or more of first central longitudinal coil axis 191A and second central longitudinal coil axis 191B, e.g., to a deployed configuration. Such expansion can enable coil structure 190 to position electrodes 170 into apposition with a blood vessel wall (e.g., for delivering electrical stimulation therapy to tissue of a patient proximate the blood vessel and/or sensing a patient parameter from a location within the blood vessel).

First coil portion 132 and second coil portion 134 can include and/or define respective coil parameters. As illustrated in the example of FIG. 3A, first coil portion 132 defines a first pitch P1 and a first coil diameter D1. Second coil portion 134 defines a second pitch P2 and a second coil diameter D2. First pitch P1 and second pitch P2 may define spacing between adjacent loops (e.g., spacing between axial midpoints of adjacent loops) of each of first coil portion 132 and second coil portion 134, respectively. First coil diameter D1 and second coil diameter D2 may be maximum outer cross-sectional dimensions of each of first coil portion 132 and second coil portion 134, respectively.

The coil parameters (e.g., at least pitch and sizing) of first coil portion 132 and second coil portion 134 can be any suitable values. First coil diameter D1 and/or second coil diameter D2 can be about 0.3 inches to about 0.9 inches (and/or any values or range of values therebetween), such as about 0.65 inches. In some examples, first coil diameter D1 and/or second coil diameter D2 is 0.65 inches. Such sizing of first coil portion 132 and second coil portion 134 can be larger (e.g., such as by about 10%, or more) than a blood vessel in which coil structure 190, including first coil portion 132 and second coil portion 134. For example, an inner diameter (ID) of the blood vessel (e.g., blood vessel 120 in the example of FIG. 3D) in which coil structure 190 is positioned can be about 0.5 inches to 0.6 inches (and/or any values or range of values therebetween), such as about 0.55 inches. Such sizing can ensure that coil structure 190, including one or more of first coil portion 132 and second coil portion 134 can firmly anchor endovascular device 160 within the blood vessel.

First pitch P1 and second pitch P2 can be about 0.01 inches to about 0.05 inches (and/or any values or range of values therebetween), such as about 0.03 inches. Such pitches can enable coil structure 190 to position electrodes 170 at suitable axially and circumferentially spaced apart locations.

First coil diameter D1 and/or first pitch P1 can be constant over at least a portion (e.g., the entirety of) first coil portion 132 (e.g., over a portion or all of the axial length of first coil portion 132, as measured in the x-axis direction according to the orthogonal x-y-z axes of FIG. 3A). Second coil diameter D2 and/or second pitch P2 can be constant over at least a portion (e.g., the entirety of) second coil portion 134 (e.g., over a portion or all of the axial length of second coil portion 134, as measured in the x-axis direction according to the orthogonal x-y-z axes of FIG. 3A). In other examples, first coil diameter D1 and/or first pitch P1 of first coil portion 132 can vary over the axial length of first coil portion 132. Additionally or alternatively, in some examples, second coil diameter D2 and/or second pitch P1 of second coil portion 134 can vary over the axial length of second coil portion 134.

Although the example of FIG. 3A and FIG. 3D illustrated coil structure 190, including first coil portion 132 and second coil portion 134, as a tubular or substantially tubular coil, other shapes and/or form factors are contemplated. For example, in some examples, coil structure 190 defines a non-tubular shape (e.g., such that a maximum cross-sectional dimension of coil structure 190 at one or more locations is not necessarily circular). In some examples, one or more of first coil portion 132 and/or second coil portion 134 define a non-constant coil diameter. In some examples, one or more of first coil portion 132 and/or second coil portion 134 define a tapering spiral (e.g., that can taper between a larger coil diameter and a smaller coil diameter).

In some examples, first coil portion 132 and second coil portion 134 of coil structure 190 define a continuous or substantially continuous coil shape. For example, first pitch P1 and second pitch P1 can be the same (e.g., equal) or substantially the same (e.g., to the extent permitted by manufacturing tolerances). In some examples, first coil diameter D1 and second coil diameter D2 are the same (e.g., equal) or substantially the same (e.g., to the extent permitted by manufacturing tolerances). Such equal or substantially equal coil pitches and coil diameters of each of first coil portion 132 and second coil portion 134 can enable coil structure 190 to have a continuous and/or the substantially continuous coil shape. Such continuous and/or the substantially continuous coil shape of coil structure 190 can be relatively easier to transform to a relatively low-profile delivery configuration, which can facilitate easier delivery and/or retrieval of endovascular device 160 and/or coil structure 190. Additionally or alternatively, the relatively simple, continuous shape of the coil formed by first coil portion 132 and second coil portion 134 of coil structure 190 (e.g., as compared to other expandable anchoring structures, such as stents and/or stent-like structures) can enable relatively simpler, easier, and/or cheaper (e.g., less costly) manufacturing and/or assembly.

In some examples, the physical configuration of each of first coil portion 132 and second coil portion 134 can be different. In some examples, the first coil portion 132 and second coil portion 134 can include and/or define one or more of a different shape, form factor, material, and/or other characteristics. For example, each first coil portion 132 and second coil portion 134 can be configured differently according to the complimentary, respective functions of each of first coil portion 132 and second coil portion 134. For example, second coil portion 134 can be configurated primarily for anchoring endovascular device 160 (e.g., as compared to first coil portion 132). First coil portion 132 can be configured primarily for carrying electrodes 170 and/or for positioning electrodes 170 into apposition with vessel wall 122 of blood vessel 120 (e.g., as illustrated in FIG. 3D). However, first coil portion 132 can be configuration to assist in anchoring endovascular device 160. Given that the second coil portion 134 does not any electrodes 170 (or any other related components), second coil portion 134 can be configured primarily for an anchoring function as compared to first coil portion 132.

In some examples, first coil portion 132 and second coil portion 134 are configured exert a different anchoring force (e.g., a force directed radially outward from first central longitudinal coil axis 191A and/or second central longitudinal coil axis 191B). For example, in some examples, second coil portion 134 is configured to provide a greater anchoring force as compared to first coil portion 132. In some examples, one or more of first coil portion 132 and second coil portion 134 are configured to exert an anchoring force (e.g., a maximum anchoring force) of about 0.005 grams to about 0.02 grams (and/or any values or range of values therebetween), such as about 0.0118 grams.

In some cases, properties of second coil portion 134 (e.g., including pitch, diameter, length) can be tuned (e.g., selected) based on one or more conditions (e.g., including the type of blood vessel in which second coil portion 134 is positioned, blood flow conditions, type of therapy and/or sensing, or the like). For example, second coil portion 134 can be selected from a kit of different second coil portions having different properties and paired with a given medical lead (e.g., elongated body 162 having one or more electrodes 170), such that a medical lead (e.g., elongated body 162 having one or more electrodes 170) can be configured to be positioned in multiple different types of blood vessel and/or accommodate different conditions based on the selectable properties of second coil portion 134.

In some examples, first pitch P1 of first coil portion 132 and second pitch P2 of second coil portion 134 are different. For example, second pitch P2 of second coil portion 134 may be less than first pitch P1 of first coil portion 132 (e.g., by any suitable amount, such as by 5%, 10%, or more). As second coil portion 134 does not carry any of electrodes 170, and thus does not provide axial and/or circumferential spacing between adjacent ones of electrodes 170, second coil portion 134 can, in some examples, define a relatively tighter coil as compared to first coil portion 132 (e.g., such that second pitch P2 of second coil portion 134 is less than first pitch P1 of first coil portion 132). In some examples, the tighter coil of second coil portion 134 (e.g., the relatively lower second pitch P2 of second coil portion 134) can enable relatively greater anchoring ability of second coil portion 134 as compared to first coil portion 132. For example, second coil portion 134 may be configured with relative more coil turns in an equal or smaller axial space as compared to first coil portion 132, which may enable a relatively greater anchoring ability of second coil portion 134 as compared to first coil portion.

In some examples, second coil diameter D2 of second coil portion 134 and first coil diameter D1 of first coil portion 132 are different. For example, second coil diameter D2 of second coil portion 134 can be greater than first coil diameter D1 of first coil portion 132. Second coil diameter D2 of second coil portion 134 can be greater than first coil diameter D1 of first coil portion 132 by any suitable amount (e.g., greater by at least 5%, 10%, or any suitable amount). In some cases, the greater diameter of second coil portion 134 can enable second coil portion 134 to provide a greater anchoring force (e.g., a force radial outward toward a blood vessel wall) as compared to first coil portion 132.

In some cases, only a portion of second coil portion 134 defines a larger coil diameter (e.g., second coil diameter D2) as compared to first coil portion 132 (e.g., first coil diameter D1). For example, in some examples, only a distal portion of second coil portion 134 defines a relatively larger coil diameter as compared to first coil portion 132. Spacing the larger coil diameter portion of second coil portion 134 from first coil portion 132 can minimize or eliminate the possibility of second coil portion 134 lifting a blood vessel wall (e.g., blood vessel wall 122 of blood vessel 120 illustrated in FIG. 3D) away from electrodes 170 positioned on first coil portion 132. In such examples, a more proximal portion of second coil portion 134 (e.g., the portion closer to first coil portion 132) can define a coil diameter equal to or substantially equal to first coil portion 132.

Second coil portion 134 can have any suitable configuration to help anchor endovascular device 160, including coil structure 190, within blood vessel 120. In some examples, second coil portion 134 includes at least a suitable number of coil loops (e.g., wherein one coil loop includes a full, 360 degree rotation around second central longitudinal coil axis 191B). In some examples, second coil portion 134 includes at least two coil loops (e.g., two full rotations around second central longitudinal coil axis 191B). However, second coil portion 134 can include fewer or more coil loops (e.g., one coil loop, three coil loops, four coil loops, five coil loops, or more). The number of coil loops of coil structure 190, including the number of coil loops of second coil portion 134, can correspond to the ability of coil structure 190 to keep endovascular device, including electrodes 170, anchoring in a stable location within vasculature of a patient (e.g., within blood vessel 120 as illustrated in FIG. 3D). First coil portion 132 can also include any suitable number of coil loops (e.g., one coil loop, two coil loops, three coil loops, four coil loops, five coil loops, or more). The number of coil loops of first coil portion 132 can correspond to the number and/or spacing (e.g., axial spacing and/or circumferential spacing) of electrodes 170 carried by first coil portion 132. In some examples, second coil portion 134 includes the same number of coil loops as first coil portion 132. In other examples, second coil portion 134 includes a different number of coil loops as first coil portion 132.

Second coil portion 134 of coil structure 190 can include any suitable configuration and/or features to enable second coil portion 134 to help anchor endovascular device 160 with vasculature of a patient (e.g., within blood vessel 120, as illustrated in the example of FIG. 3D). While first coil portion 132 may facilitate some anchoring of endovascular device 160, first coil portion 132 may, in some cases, not provide sufficient anchoring by itself such as to prevent movement (e.g., axial movement and/or circumferential movement) of endovascular device 160 within blood vessel 120. Second coil portion 134 of coil structure 190 can be configured to provide additional and/or complimentary anchoring as compared to first coil portion 132 of coil structure 190. While each of first coil portion 132 and second coil portion 134 may provide anchoring (e.g., force directed to and/or engagement with vessel wall 122 of blood vessel 120 in the example of FIG. 3D), second coil portion 134 can be configured to provide greater anchoring ability relative to first coil portion 132.

In some examples, as illustrated in the example of at least FIG. 3A, second coil portion 134 of coil structure 190 includes (e.g., is formed from) a coiled wire 192. In the example of FIG. 3A, coiled wire 192 extends between a proximal end (e.g., second coil portion proximal end 194) and a distal end (e.g., second coil portion distal end 196). In some examples, second coil portion 134 includes coiled wire 192 such that second coil portion 134 is a coiled coil (e.g., such that second coil portion 134 together with coiled wire 192 defines a coiled coil). That is, coiled wire 192 of second coil portion 134 may be a continuous wire that is coiled. Coiled wire 192 is then arranged in a coiled manner (e.g., serpentine or twisting manner) to form a coiled coil, where the coils of the wire form coiled wire 192, and the serpentine or twisting of the coiled wire forms second coil portion 134, which is a coiled coil of wire.

In some cases, using coiled wire 192 that forms a coil for anchoring endovascular device 160 and electrodes 170 within the vasculature of a patient can facilitate relatively stronger anchoring, e.g., as compared to just a having a coil portion the main body portion of endovascular device 160 (e.g., elongated body 162 of endovascular device 160, as illustrated in FIG. 3B), which can have a relatively smooth outer surface. For example, coiled wire 192 may facilitate relatively greater engagement (e.g., contact, friction, and/or the like) with vessel wall 122 (e.g., illustrated in FIG. 3D) causing endovascular device 160 to be relatively more firmly anchored within as compared to other anchoring mechanisms.

Coiled wire 192 can have any suitable configuration. Coiled wire 192 can defined any suitable outer cross-sectional dimension (e.g., diameter), such as about 0.002 inches to about 0.004 inches (and/or any values or range of values therebetween), such as about 0.003 inches. In some examples, a diameter of coiled wire 192 is 0.0032 inches. In some examples, coiled wire 192 defines a constant or substantially constant outer cross-sectional dimension (e.g., diameter), such as along the entire length of coiled wire 192 (e.g., between the proximal end and distal end of coiled wire 192).

In some examples, coiled wire 192 is configured to receive a guidewire, a stylet, and/or a straightening element therethrough (e.g., through the radial center of the coil defined by coiled wire 192). For example, the coil defined by coiled wire 192 can define a lumen configured to receive an elongated structure (e.g., a guidewire, a stylet, and/or a straightening element). In some examples, a tubular body (e.g., a polymer tubular body) is provided within the lumen of the coil formed by coiled wire 192. The tubular body can define the channel (e.g., lumen) through which the elongated structure (e.g., a guidewire, a stylet, and/or a straightening element) is received. In some examples, lumen 152 (e.g., which can extend through at least first coil portion 132) is continuous with the lumen of second coil portion 134 (e.g., the coil formed by coil wire 192). In some examples, endovascular device 160 is configured to receive an elongated structure (e.g., a guidewire, a stylet, and/or a straightening element) such that the elongated body can extend distally of second coil portion distal end 196.

First coil portion 132 and second coil portion 134 can have any suitable relative orientation and/or positioning. In some examples, coiled wire 192 that forms second coil portion 134 extends distally of distal end of first coil portion 132 (e.g., distally of elongated body distal end 164). In some examples, a portion of coiled wire 192 that forms second coil portion 134 is mechanically coupled to a portion of first coil portion 132. A proximal end of coiled wire 192 (e.g., second coil portion proximal end 194, which may be a proximalmost end of coiled wire 192) can be mechanically coupled (e.g., fixedly mechanically coupled) to a distal end of first coil portion 132 (e.g., elongated body distal end 164). First coil portion 132 (e.g., which can include a portion of elongated body 162) and second coil portion 134 (e.g., including coiled wire 192) can be mechanically coupled via one or more of molding (e.g., injection molding), crimping, adhesive, and/or another suitable mechanical coupling mechanism. For example, a suitable material (e.g., a polymer material) can be molded over (e.g., overmolded) over at least a portion of each of first coil portion 132 (e.g., which can include a portion of elongated body 162) and second coil portion 134 (e.g., including coiled wire 192) to mechanically couple these respective coil portions.

In other examples, first coil portion 132 and second coil portion 134 can be mechanically coupled such that first coil portion 132 and second coil portion 134 are at least partially overlapping (e.g., partially overlapping along the axial length of each of first coil portion 132 and second coil portion 134).

Coiled wire 192 of second coil portion 134 can include any suitable material and/or combination of materials. In some examples, coiled wire 192 is a bare metal wire. In some examples, coiled wire 192 does not include a polymer (e.g., such that second coil portion 134 does not include a polymer). In some examples, coiled wire 192 includes a shape memory material (e.g., nitinol). A shape-memory material can enable coiled wire 192 to be at least partially or fully self-expanding (e.g., such as to, with reference to FIG. 3D, engage blood vessel wall 122 of blood vessel 120 to anchor endovascular device 160 within blood vessel 120). In some examples coiled wire 192 defines a pre-shaped coil (also referred to a pre-formed coil shape). In some examples, coiled wire 192 is configured to assume a predetermined coil shape in the absence of external forces (e.g., after deployment of coiled wire 192 and/or second coil portion 134 from a delivery catheter or another physically constraining body). The pre-shaped coil shape formed by coiled wire 192 can include one or both of the coil shape of second coil portion 134 (e.g., that extends around second central longitudinal coil axis 191B, as shown in the example of FIG. 3A and FIG. 3D) and the coil shape of coiled wire 192 (e.g., that extends around elongated body central longitudinal axis 161, as illustrated in the example of FIG. 3C).

In some examples, coiled wire 192 includes an antithrombogenic coating. In some examples, coiled wire 192 includes a surface texture treatment (e.g., a laser treatment, which can facilitate endothelialization of coiled wire 192).

In some examples, coiled wire 192 includes one or more materials configured to be relatively mechanically robust and/or fatigue resistant. In some examples, coiled wire 192 includes one or more metal materials. For example, coiled wire 192 can include one or more metal alloys, such as one or more of platinum-iridium (e.g., Pt-20Ir), nickel-cobalt, titanium-tantalum-tin (TiTaSn), platinum-tungsten (Pt—W), other stainless steel or nickel-based alloys (e.g., MP35N), and/or beta-titanium alloys (also referred to as Beta Ti alloys), such as Ti-15Mo. Use of such materials may enable use of a relatively thinner wire that that forms coiled wire 192, which may facilitate relatively easier delivery and/or navigation of endovascular device 160 through vasculature of a patient. Additionally, use of such materials can enable implantation of endovascular device 160 within a blood vessel (e.g., blood vessel 120 of FIG. 3D) over relatively longer periods of time with minimal risk of mechanical failure (e.g., mechanical failure that would compromise the ability of coiled wire 192 to anchor endovascular device 160 within blood vessel 120). Use of such materials can also enable coiled wire 192 to form a pre-shaped coil shape.

In some examples, coiled wire 192 includes a radiopaque or radiographic material. In some examples, coiled wire 192 additionally or alternatively includes one or more radiopaque or radiographic markers positioned on coiled wire 192 (e.g., multiple markers spaced apart along coiled wire 192 at respective axial and/or circumferential positions on coiled wire 192). In examples in which coiled wire 192 includes one or more radiopaque or radiographic materials or markers, a user (e.g., a clinician) may be able to visualize coiled wire 192 via a suitable medical imaging modality (e.g., x-ray, fluoroscopy, angiography, and/or the like). Visualization of coiled wire 192 via the suitable medical imaging modality can enable a clinician to determine a shape, position, and/or other information about coiled wire 192. For example, visualization of coiled wire 192 may enable a clinician to determine a shape, position, and/or other information about coiled wire 192 relative to other structural features of endovascular therapy system 100 and/or anatomical features of a patient (e.g., patient 12 in the example of FIG. 1).

Coiled wire 192 can have any suitable configuration. In some examples, as illustrated in the example of FIG. 3C, coiled wire 192 can define a coil extending around elongated body central longitudinal axis 161. In some examples, the coil formed by coiled wire 192 defines a third pitch P3 and a third coil diameter D3. Third pitch P3 may define spacing between adjacent loops (e.g., spacing between axial midpoints of adjacent loops) of the coil formed by coiled wire 192. Third coil diameter D3 may be maximum outer cross-sectional dimensions of the coil formed by coiled wire 192. Coil diameter D3 can be about 0.02 inches to about 0.04 inches (and/or any values or range of values therebetween), such as about 0.03 inches. In some examples, coil diameter D3 is 0.027 inches. Third pitch P3 of coiled wire 192 can be about 0.002 inches to about 0.004 inches (and/or any values or range of values therebetween), such as about 0.003 inches. In some examples, third pitch P3 of the coil formed by coiled wire 192 is 0.0032 inches.

As coiled wire 192 itself forms the larger coiled shape of second coil portion 134, each of third pitch P3 and third coil diameter D3 of coiled wire 192 may be significantly less than second pitch P2 and second coil diameter D2 defined by second coil portion 134. Similarly, each of third pitch P3 and third coil diameter D3 of coiled wire 192 may be less than first pitch P1 and first coil diameter D1 defined by first coil portion 132. In some examples, third pitch P3 of coiled wire 192 is less (e.g., substantially less than, such as by at least 50%) one or more of first pitch P1 of first coil portion 132 and second pitch P2 of second coil portion 134. In some examples, third coil diameter D3 of coiled wire 192 is less than (e.g., substantially less than, such as by at least 50%) one or more of first coil diameter D1 of first coil portion 132 and second coil diameter D2 of second coil portion 134.

In some examples, as illustrated in the example of FIG. 3B, endovascular therapy system 100 includes a plurality of conductor wires 180. Conductor wires 180 can be configured to electrically connect electrodes 170 to a medical device (e.g., medical device 14 of FIG. 1). Each of conductor wires 180 can extend along (e.g., within) at least a portion of elongated body 162 of endovascular device 160. In some examples, conductor wires 180 form a multi-filar coil within at least a portion of endovascular device 160 (e.g., within at least a portion of elongated body 162). In some examples, some or all of conductor wires 180 are part of endovascular device 160, while in other examples, some or all of conductor wires 180 are separate components from endovascular device 160.

In some examples, as discussed further with respect to FIG. 4A and FIG. 4B, coiled wire 192 can be at least partially coextensive with one or more of conductor wires 180. For example, rather than a proximal end of coiled wire 192 being mechanically coupled to elongated body distal end 164, at least a portion of coiled wire 192 can be coextensive with (e.g., extend within) first coil portion 132, such that coiled wire 192 is at least partially coextensive with one or more of conductor wires 180. In such examples, at least a portion of coiled wire 192 (e.g., at least a proximal portion of coiled wire 192 including a proximal end of coiled wire 192) can extend proximally of first coil portion 132.

In some examples, endovascular device 160 is configured to be at least partially introduced into, positioned in, and/or implanted within vasculature (e.g., blood vessel 120 as illustrated in FIG. 3D) of a patient (e.g., patient 12 of FIG. 1). Endovascular device 160 may include an electrically insulative material covering at least some portions of endovascular device 160 (e.g., one of the materials listed above). The electrically insulative material covering at least some portions of endovascular device 160 can electrically insulate elements disposed within endovascular device 160 (e.g., electrically insulate electrically conductive components, such as conductor wires 180, from blood or other tissue, such as when endovascular device 160 is positioned in or advanced through a blood vessel of patient 12).

In some examples, at least a portion of each of conductor wires 180 are housed by the insulative material of elongated body 162 of endovascular device 160. For example, each of conductor wires 180 can extend within a lumen of endovascular device 160 (e.g., within elongated body 162 of endovascular device 160, as illustrated in FIG. 3B). In some examples, an electrically insulative material of endovascular device 160 can be configured to electrically insulate portions of conductor wires 180 that run along the length of endovascular device 160. In some examples, each of conductor wires 180 extends along at least a portion of coil structure 190 (e.g., along at least a portion of first coil portion 132).

Some or all of conductor wires 180 may include a material or combination of materials configured to facilitate relatively high flexibility, high axial extensibility, and/or high fatigue resistance. For example, one or more wires of conductor wires 180 includes a beta-titanium alloy (also referred to as Beta Ti alloys). In some examples, the beta-titanium alloy comprises a Ti-15Mo alloy. Certain beta-titanium alloys, including Ti-15Mo alloy and similar titanium alloys enable higher wire count coils (e.g., twelve wire or greater, including equal to or greater than sixteen wire coils), such as for situations in which a relatively high number of individually controlled electrodes are needed in a small space including nerve stimulation and/or sensing from endovascular locations.

In some examples, each of conductor wires 180 are electrically connected to a respective electrode of electrodes 170A-170D. In some examples, each of electrodes 170A-170D is configured to receive and/or otherwise mechanically couple to one or more conductor wires of conductor wires 180 (e.g., to facilitate the electrical connection between each of conductor wires 180 and one or more of electrodes 170). Each of electrodes 170 can include an electrical contact portion configured to facilitate electrical connection to conductor wires 180.

In some examples, more than one of electrodes 170 are electrically connected to a common conductor wire of conductor wires 180 (e.g., some of electrodes 170 can be “shorted” together). For example, one of conductor wires 180 can be configured to connect to a least a first electrode and a second electrode of electrodes 170 (e.g., such that a medical device 14 can simultaneously control each of the first electrode and the second electrode of electrodes 170 together). Shorting of at least some of electrodes 170 can facilitate control of multiple electrodes at the same time (e.g., for delivery of electrical stimulation therapy and/or sensing).

As discussed above, and as illustrated in FIG. 3D, electrodes 170 may be configured to deliver electrical stimulation therapy to tissue or sense a patient parameter (e.g., a signal, including a bioelectric signal) from a location within blood vessel 120. For example, electrodes 170 can be sized, shaped, and/or otherwise configured to transmit (e.g., deliver) and/or receive electrical signals. Electrodes 170 can include a suitable electrically conductive material (e.g., TiTaSn).

Endovascular therapy system 100 can include any suitable number of electrodes 170 for delivery of stimulation therapy (e.g., electrical stimulation therapy) and/or sensing from an endovascular location. While the example of FIG. 3A illustrates endovascular therapy system as including four of electrodes 170, endovascular therapy system 100 can include any suitable number of electrodes 170 (e.g., one electrode, two electrodes, three electrodes, four electrodes, five electrodes, six electrodes, seven electrodes, eight electrodes, nine electrodes, ten electrodes, twelve electrodes, fifteen electrodes, twenty electrodes, thirty electrodes, or more). Each of electrodes 170 can be disposed at respective spaced-apart locations along and/or around coil structure 190.

With reference to at least FIG. 3A, endovascular device 160 can include electrodes 170 disposed on, carried by, or otherwise defined by a portion of endovascular device 160 (e.g., elongated body 162 of endovascular device 160). As illustrated in the example of FIG. 3A, each of electrodes 170A-170D are disposed at respective, spaced apart along positions along distal portion 150 of endovascular device 160 (e.g., along a portion of elongated body 162 that defines first coil portion 132). The number and spacing of electrodes 170 may correspond to the therapy being delivered and/or type of sensing, the implant location (e.g., the specific blood vessel), or to account for specific patient factors (e.g., biological indicator such as age or gender, a disease state, a blood pressure, and/or a blood velocity).

Electrodes 170 can have any suitable positioning and/or spacing relative to each other and/or endovascular device 160. In some examples, and with reference to the example of FIG. 3A, electrodes 170A-170D are axially spaced apart from each other along first central longitudinal coil axis 191A (e.g., such that axially adjacent electrodes of electrodes 170A-170D are axially spaced apart along first central longitudinal coil axis 191A at respective axial locations). In some examples, as illustrated in the example of FIG. 3D, each of electrodes 170A-170D faces in a unique radial direction (e.g., a unique radial direction outward from first central longitudinal coil axis 191A).

For example, as illustrated in the example of FIG. 3D, electrode 170A is positioned on coil structure 190 of endovascular device 160 (e.g., on first coil portion 132) such that electrode 170A generally faces in a first radial direction 171A (e.g., which may be a first radial direction facing radially outward from first central longitudinal coil axis 191A). Electrode 170B is positioned on coil structure 190 of endovascular device 160 (e.g., on first coil portion 132) such that electrode 170B generally faces in a second radial direction 171B (e.g., which may be a second radial direction facing radially outward from first central longitudinal coil axis 191A). Electrode 170C is positioned on coil structure 190 of endovascular device 160 (e.g., on first coil portion 132) such that electrode 170C generally faces in a third radial direction 171C (e.g., which may be a third radial direction facing radially outward from first central longitudinal coil axis 191A). Electrode 170D is positioned on coil structure 190 of endovascular device 160 (e.g., on first coil portion 132) such that electrode 170D generally faces in a fourth radial direction 171D (e.g., which may be a fourth radial direction facing radially outward from first central longitudinal coil axis 191A). Each of first radial direction 171A, second radial direction 171B, third radial direction 171C, and fourth radial direction 171D can be different radial directions (e.g., relative to first central longitudinal coil axis 191A).

In other examples, at least some of electrodes 170A-170D face in a common radial direction outward from first central longitudinal coil axis 191A. For example, two or more of electrodes 170A-170D can be positioned on coil structure 190 of endovascular device 160 (e.g., on first coil portion 132) such that two of more of electrodes face in a common radial rejection relative to first central longitudinal coil axis 191A (e.g., at least one of first radial direction 171A, second radial direction 171B, third radial direction 171C, and fourth radial direction 171D). Aligning one or more electrodes 170 to face in a common a common radial direction can be used in examples in which target tissue (e.g., one or more nerves) is located at a particular circumferential position relative to the blood vessel in which endovascular device 160 is positioned. For example, targeting of a vagus nerve outside of a suitable blood vessel (e.g., a jugular artery or carotid vein) may be accomplished by two or more of electrodes 170 that are configured to face in a common radial direction outward from the suitable blood vessel.

Although FIG. 3A is described with respect to electrodes 170 that are configured to deliver electrical stimulation therapy and/or sense electrical signals, endovascular therapy system 100 can additionally or alternatively include other types of therapy delivery elements and/or sensors. In some examples, endovascular therapy system 100 includes one or more ultrasound transducers, chemical delivery elements (e.g., fluid delivery elements and/or drug elution elements) which can be configured to be attached to coil structure 190 using a similar method of attachment as electrodes 170. In some examples, endovascular therapy system 100 additionally or alternatively includes one or more temperature sensors, pressure sensors, optical sensors, impedance sensors, chemical sensors, and/or other suitable types of sensors, which can be configured to be attached to coil structure 190 using a similar method of attachment as electrodes 170.

FIG. 4A and FIG. 4B illustrate example endovascular therapy system 400 including an example endovascular device 460, which is an example of endovascular device 16 of FIG. 1 or endovascular device 160 of at least FIG. 3A. Endovascular device 460 can be configured similar to endovascular device 160 of FIG. 3A, FIG. 3B, FIG. 3C, and/or FIG. 3D except as described herein. For example, endovascular device 460 includes an elongated body 462 with a coil structure 490 at a distal portion 450 of elongated body 462, wherein each of elongated body 462, coil structure 490, and distal portion 450 can be examples of and/or configured similarly to elongated body 162, coil structure 190, and distal portion 150, respectively, except as described herein. Endovascular therapy system 400 includes electrode 470A, electrode 470B, electrode 470C, and electrode 470D, collectively referred to herein as electrodes 470, which may be examples of electrode 170A, electrode 170B, electrode 170C, and electrode 170D of at least FIG. 3A, respectively.

FIG. 4B illustrates a cross-sectional view of a portion of endovascular therapy system 400 of FIG. 4A. In the example of FIG. 4B, the cross-section is taken through the B-B section lines of FIG. 4A and face in the positive x-axis direction according to the orthogonal x-y-z axes of FIG. 4A.

As illustrated in FIG. 4B, endovascular therapy system 400 includes a plurality of conductor wires 480. Conductor wires 480 can be configured to electrically connect electrodes 470 to a medical device (e.g., medical device 14 of FIG. 1). Conductor wires 480 can be configured similar to conductor wires 180 of at least FIG. 3B, except as described herein.

In the example of FIG. 4A, elongated body 462 of endovascular device 460 defines an elongated body central longitudinal axis 461 extending along endovascular device 460. Elongated body central longitudinal axis 461 may be a central longitudinal axis of one or more of elongated body 462 and/or endovascular device 460. Elongated body 462 can be a tubular body defining at least one lumen (e.g., a lumen 452 as illustrated in the example of FIG. 4B).

As discussed with respect to coil structure 190 of at least FIG. 3A, coil structure 490 can include multiple portions having different characteristics (e.g., different shape, form factors, materials, and/or other characteristics). For example, as illustrated in at least FIG. 4A, coil structure 490 of endovascular device 460 includes at least a first coil portion 432 and a second coil portion 434. First coil portion 432 and second coil portion 434 can be configured similarly to first coil portion 132 and second coil portion 134 of at least FIG. 3A, except as described herein.

In the example of FIG. 4A, first coil portion 432 defines a first central longitudinal coil axis 491A. First central longitudinal coil axis 491A may be an example of first central longitudinal coil axis 191A of FIG. 3A, except as described herein. Second coil portion 434 defines a second central longitudinal coil axis 491B. Second central longitudinal coil axis 491B may be an example of second central longitudinal coil axis 191B of FIG. 3A, except as described herein.

In the example of FIG. 4A and FIG. 4B, second coil portion 434 includes a coiled wire 492. Coiled wire 492 can be configured similarly to coiled wire 192 of at least FIG. 3A, except as described herein. In the example of FIG. 4A, coiled wire 192 extends between a proximal end (not shown in the example of FIG. 4A) and coiled wire distal end 496. Coiled wire distal end 496 may be a distalmost end of coiled wire 492 and/or endovascular device 460.

As illustrated in the example of FIG. 4A and FIG. 4B, coiled wire 492 can be at least partially coextensive with elongated body 462 of endovascular device 460. In some examples, coiled wire 492 can extend along (e.g., within) within first coil portion 432 (e.g., which is defined by elongated body 462). For example, coiled wire 492 can be coextensive with one or more of conductor wires 480 that extend within the endovascular device 460 (e.g., that extend within first coil portion 432). Coiled wire 492 can extend alongside conductor wires 480 within first coil portion 432. In such cases, a proximal end of coiled wire 492 can extend proximally of coil structure 490, such a proximal end of endovascular device 460.

In some examples, at least a portion of coiled wire 492 and conductor wires 480 together form a multi-filar coil. For example, the portion of coiled wired 492 extending within elongated body 462 and conductor wires 480 can form a multi-filar coil. A multi-filar coil configuration of coiled wire 492 and conductor wires 480 can be relatively more mechanically robust as compared to other configurations (e.g., configurations in which coiled wire 492 and conductor wires 480 extend parallel to each other within elongated body 462 in a non-coiled configuration).

As described above, each of conductor wires 480 can be electrically coupled to, and terminate at, a respective one of electrodes 470A-470D. Once each conductor wire 480 terminates at a respective one of electrodes 470A-480D, coiled wire 492 can extend distally of electrodes 470 and elongated body distal end 464 of elongated body 462. The portion of coiled wire 492 that extends distally of elongated body 462 can form second coil portion 434, as illustrated in the example of FIG. 4A. For example, coiled wire 492 can extend distally of elongated body distal end 464 of elongated body 462 (e.g., which may be a distalmost end of elongated body 462) such that coiled wire distal end 496 of coiled wire 492 is distal to elongated body distal end 464 of elongated body 462.

In some cases, having coiled wire 492 coextensive with at least a portion of elongated body 462 obviates the mechanical bond between coiled wire 492 and a distal portion (e.g., which can include elongated body distal end 464) of elongated body 462. Such a configuration can be relatively more mechanically robust as compared to configurations in which first coil portion 432 and the second coil portion 434 (e.g., formed by coiled wire 492) are mechanically coupled together at a junction between the first coil portion 432 and the second coil portion 434, e.g., as illustrated in, and discussed with respect to, the example of FIG. 3A.

In the example of FIG. 4A, coiled wire 492 is not configured for electrical stimulation therapy and/or sensing (e.g., is not connected to a medical device, such as medical device 14 of FIG. 1). In other examples, coiled wire 492 can be mechanically and/or electrically connected to a medical device and configured to deliver electrical stimulation therapy and/or for configured to receive signals (e.g., bioelectric signals). For example, because coiled wire 492 is at least partially coextensive with elongated body 462 and one or more of conductor wires 480, coiled wire 492 can be configured to connect to a medical device (e.g., medical device 14 of FIG. 1) in a similar manner as conductor wires 480.

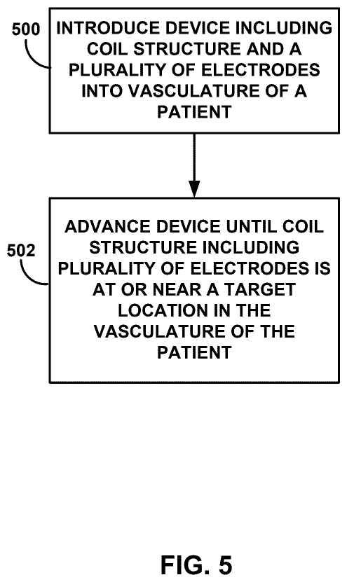

FIG. 5 is a flow diagram illustrating an example technique for using an endovascular therapy system including an endovascular device according to the techniques of this disclosure, which may include placing an endovascular device (e.g., which may be and/or include a medical lead) adjacent a target location in vasculature of a patient. The technique of FIG. 5 is described with respect to therapy system 10 of FIG. 1, as well as endovascular therapy system 100 of at least FIG. 3A (which is an example of therapy system 10 of FIG. 1), but may be used with any of the device, systems, and/or elements of systems described in this disclosure.

In the example of FIG. 5, the technique includes introducing an endovascular device (e.g., endovascular device 16 and/or endovascular device 160) into vasculature of patient 12 (500). For example, a user (e.g., a clinician) may introduce at least distal portion 15 of endovascular device 16 through an access point in patient 12 including a femoral artery access point or radial artery access point. In some examples, one or more of an introducer sheath, a guide catheter, and/or a guidewire is used to facilitate introduction of endovascular device 16 into patient 12.

In the example of FIG. 5, the technique further includes advancing endovascular device 16 through the vasculature of patient 12 until electrodes 17 are adjacent a target location in the vasculature of patient 12 (502). In some examples, a clinician advances endovascular device 16 through vasculature of patient 12 until electrodes 17 are located within a cranial blood vessel proximate one or more target brain structures. In other examples, such as in cases of vagus nerve stimulation and/or sensing, a clinician advances endovascular device 16 until electrodes 17 are positioned with a suitable blood vessel (e.g., jugular vein) and positioned adjacent a vagus nerve.

In some examples, the method includes causing coil structure 19 (e.g., and/or coil structure 190) to transform from the delivery (e.g., compressed, relatively low profile, and/or the like) configuration to the deployed (e.g., expanded) configuration, e.g., once electrodes 17 are adjacent the target site. In the deployed configuration of coil structure 19, one or more of electrodes 17 can be positioned into apposition with the vessel wall (e.g., the vessel wall 122 of blood vessel 120 as illustrated in the example of FIG. 3D). Causing coil structure 19 to transform from the delivery configuration to the deployed configuration can include removing a straightening element from endovascular device 16 (e.g., such as a wire that causes coil structure 19 to assume an uncoiled shape and/or a relatively lower profile shape). Additionally or alternatively, causing coil structure 19 to transform from the delivery configuration to the deployed configuration can include advancing coil structure 19 distally of a sheath or other elongated body surrounding at least coil structure 19. Once deployed, at least a portion of coil structure 19 (e.g., first coil portion 132 and/or second coil portion 134 of coil structure 190) can be configured to anchor endovascular device 16, coil structure 19, and electrodes within the blood vessel (e.g., blood vessel 120).

In some examples, the method includes repositioning endovascular device 16, including coil structure 19 and electrodes 17, at a different location. For example, a clinician may be able to transform endovascular device 16 back to the delivery configuration (e.g., after already having transformed endovascular device 16 to the deployed configuration) to reposition endovascular device 16, including coil structure 19 and electrodes 17, at a different location within a blood vessel. In some examples, repositioning endovascular device 16, including coil structure 19 and electrodes 17, can include positioning the sheath or other elongated body surrounding at least coil structure 19 (e.g., re-sheathing coil structure 19) such that coil structure 19 can be transformed back to the delivery configuration and navigated to a different location within the blood vessel. Additionally or alternatively, the method can include re-inserting the straightening element back into endovascular device 16 (e.g., such that at least coil structure 19 is transformed back to the delivery configuration).

After electrodes 17 are adjacent the target location (e.g., proximate one or more brain structures, a vagus nerve, or another suitable never), the method can include initiating (e.g., via programmer 20, or another suitable device) electrical stimulation therapy and/or sensing of one or more patient parameters by medical device 14 via electrodes 17.

This disclosure includes the following non-limiting examples.

Example 1: An endovascular device includes an elongated body configured to be introduced into vasculature of a patient; and a coil structure at a distal portion of the elongated body, the coil structure including: a first coil portion configured to carry a plurality of electrodes; and a second coil portion formed from a coiled wire such that the second coil portion together with the coiled wire defines a coiled coil, the coiled wire extending distally of a distal end of the first coil portion and configured to anchor the elongated body and the coil structure within the vasculature of the patient.

Example 2: The endovascular device of example 1, wherein: the first coil portion defines a first pitch and a first coil diameter, the second coil portion defines a second pitch and a second coil diameter, and the coiled wire defines a third pitch and a third coil diameter, wherein the third pitch is less than the first pitch and less than the second pitch, and wherein the third coil diameter is less than the first coil diameter and the second coil diameter.

Example 3: The endovascular device of example 2, wherein the first pitch and the second pitch are the same.

Example 4: The endovascular device of example 2, wherein the first pitch and the second pitch are different.

Example 5: The endovascular device of any of examples 2 through 4, wherein the second coil diameter is equal to or greater than the first coil diameter.

Example 6: The endovascular device of any of examples 1 through 5, wherein the first coil portion defines a central longitudinal axis extending through a radial center of the first coil portion such that the first coil portion extends around the central longitudinal axis, and wherein axially adjacent electrodes of the plurality of electrodes are axially spaced apart along the central longitudinal axis at respective axial locations.

Example 7: The endovascular device of example 6, wherein each electrode of the plurality of electrodes faces in a unique radial direction outward from the central longitudinal axis.

Example 8: The endovascular device of example 6, wherein at least some electrodes of the plurality of electrodes face in a common radial direction outward from the central longitudinal axis.

Example 9: The endovascular device of any of examples 1 through 8, wherein the first coil portion is a continuous extension of the elongated body.

Example 10: The endovascular device of any of examples 1 through 9, wherein a proximal end of the coiled wire is mechanically coupled to a distal end of the first coil portion.

Example 11: The endovascular device of any of examples 1 through 9, wherein the coiled wire extends within the first coil portion.

Example 12: The endovascular device of example 11, wherein a plurality of conductor wires extend within the first coil portion, each of the plurality of conductor wires electrically coupled to a respective electrode of the plurality of electrodes, and wherein the coiled wire extends alongside the plurality of conductor wires within the first coil portion.

Example 13: The endovascular device of any of examples 1 through 12, wherein the first coil portion includes a polymer, and wherein the second coil portion does not include the polymer.

Example 14: The endovascular device of any of examples 1 through 13, wherein the coiled wire includes one or more of platinum-iridium, nickel-cobalt, titanium-tantalum-tin, platinum-tungsten, and beta-titanium alloys.

Example 15: The endovascular device of any of examples 1 through 14, wherein the coil structure is configured to transform from a relatively low-profile delivery configuration to a deployed configuration to position the plurality of electrodes to deliver electrical stimulation to tissue of the patient or sense a patient parameter from a location within the vasculature of the patient.

Example 16: A method includes introducing an endovascular device into vasculature of a patient, the endovascular device includes an elongated body configured to be introduced into the vasculature of the patient, and a coil structure at a distal portion of the elongated body, the coil structure including: a first coil portion configured to carry a plurality of electrodes, and a second coil portion formed from a coiled wire such that the second coil portion together with the coiled wire defines a coiled coil, the coiled wire extending distally of a distal end of the first coil portion and configured to anchor the elongated body and the coil structure within the vasculature of the patient; and advancing the endovascular device until the plurality of electrodes are at or near a target location in the vasculature of the patient.

Example 17: The method of example 16, wherein: the first coil portion defines a first pitch and a first coil diameter, the second coil portion defines a second pitch and a second coil diameter, and the coiled wire defines a third pitch and a third coil diameter, wherein the third pitch is less than the first pitch and less than the second pitch, and wherein the third coil diameter is less than the first coil diameter and the second coil diameter.

Example 18: The method of example 17, wherein the first pitch and the second pitch are the same.

Example 19: The method of example 17, wherein the first pitch and the second pitch are different.

Example 20: The method of any of examples 17 through 19, wherein the second coil diameter is equal to or greater than the first coil diameter.

Example 21: The method of any of examples 16 through 20, wherein the first coil portion defines a central longitudinal axis extending through a radial center of the first coil portion such that the first coil portion extends around the central longitudinal axis, and wherein axially adjacent electrodes of the plurality of electrodes are axially spaced apart along the central longitudinal axis at respective axial locations.

Example 22: The method of example 21, wherein each electrode of the plurality of electrodes faces in a unique radial direction outward from the central longitudinal axis.

Example 23: The method of example 21, wherein at least some electrodes of the plurality of electrodes face in a common radial direction outward from the central longitudinal axis.

Example 24: The method of any of examples 16 through 23, wherein the first coil portion is a continuous extension of the elongated body.

Example 25: The method of any of examples 16 through 24, wherein a proximal end of the coiled wire is mechanically coupled to a distal end of the first coil portion.

Example 26: The method of any of examples 16 through 24, wherein the coiled wire extends within the first coil portion.

Example 27: The method of example 26, wherein a plurality of conductor wires extend within the first coil portion, each of the plurality of conductor wires electrically coupled to a respective electrode of the plurality of electrodes, and wherein the coiled wire extends alongside the plurality of conductor wires within the first coil portion.

Example 28: The method of any of examples 16 through 27, wherein the first coil portion includes a polymer, and wherein the second coil portion does not include the polymer.

Example 29: The method of any of examples 16 through 28, wherein the coiled wire includes one or more of platinum-iridium, nickel-cobalt, titanium-tantalum-tin, platinum-tungsten, and beta-titanium alloys.

Example 30: The method of any of examples 16 through 29, wherein the coil structure is configured to transform from a relatively low-profile delivery configuration to a deployed configuration to position the plurality of electrodes to deliver electrical stimulation to tissue of the patient or sense a patient parameter from a location within the vasculature of the patient.

Example 31: An endovascular device includes an elongated body configured to be introduced into vasculature of a patient; and a coil structure at a distal portion of the elongated body, the coil structure including: a first coil portion defining a first pitch, a first coil diameter, and a central longitudinal axis extending through a radial center of the first coil portion such that the first coil portion extends around the central longitudinal axis; a second coil portion defining a second pitch and a second coil diameter, the second coil portion formed from a coiled wire such that the second coil portion together with the coiled wire defines a coiled coil, the coiled wire extending distally of a distal end of the first coil portion and configured to anchor the elongated body and the coil structure within the vasculature of the patient; and a plurality of electrodes carried by the first coil portion such that axially adjacent electrodes of the plurality of electrodes are axially spaced apart along the central longitudinal axis at respective axial locations, wherein the coiled wire defines a third pitch, the third pitch less than the first pitch and less than the second pitch, and wherein the coiled wire defines a third coil diameter, the third coil diameter less than the first coil diameter and the second coil diameter.

Example 32: The endovascular device of example 31, wherein a proximal end of the coiled wire is mechanically coupled to a distal end of the first coil portion.

Example 33: The endovascular device of example 31, wherein the coiled wire extends within the first coil portion.

The operations and techniques described in this disclosure, including those attributed to system 10, medical device 14, programmer 20, or various constituent components, may be implemented, at least in part, in hardware, software, firmware or any combination thereof. For example, various aspects of the techniques may be implemented within one or more processors, including one or more microprocessors, digital signal processors (DSPs), application specific integrated circuits (ASICs), field-programmable gate array (FPGAs), or any other equivalent integrated or discrete logic circuitry, as well as any combinations of such components, embodied in programmers, such as clinician or patient programmers, medical devices, or other devices. Processing circuitry, control circuitry, and sensing circuitry, as well as other processors and controllers described herein, may be implemented at least in part as, or include, one or more executable applications, application modules, libraries, classes, methods, objects, routines, subroutines, firmware, and/or embedded code, for example. In addition, analog circuits, components and circuit elements may be employed to construct one, some or all of the processing circuitry 30, instead of or in addition to the partially or wholly digital hardware and/or software described herein. Accordingly, analog or digital hardware may be employed, or a combination of the two.

In one or more examples, the functions described in this disclosure may be implemented in hardware, software, firmware, or any combination thereof. If implemented in software, the functions may be stored on, as one or more instructions or code, a computer-readable medium and executed by a hardware-based processing unit. The computer-readable medium may be an article of manufacture including a non-transitory computer-readable storage medium encoded with instructions. Instructions embedded or encoded in an article of manufacture including a non-transitory computer-readable storage medium encoded, may cause one or more programmable processors, or other processors, to implement one or more of the techniques described herein, such as when instructions included or encoded in the non-transitory computer-readable storage medium are executed by the one or more processors. Example non-transitory computer-readable storage media may include RAM, ROM, programmable ROM (PROM), erasable programmable ROM (EPROM), electronically erasable programmable ROM (EEPROM), flash memory, a hard disk, a compact disc ROM (CD-ROM), a floppy disk, a cassette, magnetic media, optical media, or any other computer readable storage devices or tangible computer readable media.

In some examples, a computer-readable storage medium comprises non-transitory medium. The term “non-transitory” may indicate that the storage medium is not embodied in a carrier wave or a propagated signal. In certain examples, a non-transitory storage medium stores data that can, over time, change (e.g., in RAM or cache).

The functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements.

Various examples of the disclosure have been described. Any combination of the described systems, operations, or functions is contemplated. These and other examples are within the scope of the following claims.

As used herein, relative terms such as “about,” “substantially,” and/or similar terms or phrases may indicate the exact value or nearly the exact value (e.g., to the extent permitted by manufacturing tolerances). For example, “about” can also refer to a certain percentage of the recited value (e.g., within 1%, 5%, or 10%).

What is claimed is:

1. An endovascular device comprising:
an elongated body configured to be introduced into vasculature of a patient; and
a coil structure at a distal portion of the elongated body, the coil structure including:
a first coil portion configured to carry a plurality of electrodes; and
a second coil portion formed from a coiled wire such that the second coil portion together with the coiled wire defines a coiled coil, the coiled wire extending distally of a distal end of the first coil portion and configured to anchor the elongated body and the coil structure within the vasculature of the patient.

2. The endovascular device of claim 1,
wherein:
the first coil portion defines a first pitch and a first coil diameter,
the second coil portion defines a second pitch and a second coil diameter, and
the coiled wire defines a third pitch and a third coil diameter,
wherein the third pitch is less than the first pitch and less than the second pitch, and
wherein the third coil diameter is less than the first coil diameter and the second coil diameter.

3. The endovascular device of claim 2, wherein the first pitch and the second pitch are the same.

4. The endovascular device of claim 2, wherein the first pitch and the second pitch are different.

5. The endovascular device of any of claims 2 through 4, wherein the second coil diameter is equal to or greater than the first coil diameter.

6. The endovascular device of claim 5,
wherein the first coil portion defines a central longitudinal axis extending through a radial center of the first coil portion such that the first coil portion extends around the central longitudinal axis, and
wherein axially adjacent electrodes of the plurality of electrodes are axially spaced apart along the central longitudinal axis at respective axial locations.

7. The endovascular device of claim 6, wherein each electrode of the plurality of electrodes faces in a unique radial direction outward from the central longitudinal axis.

8. The endovascular device of claim 6, wherein at least some electrodes of the plurality of electrodes face in a common radial direction outward from the central longitudinal axis.

9. The endovascular device of claim 1, wherein the first coil portion is a continuous extension of the elongated body.

10. The endovascular device of claim 1, wherein a proximal end of the coiled wire is mechanically coupled to a distal end of the first coil portion.

11. The endovascular device of claim 1, wherein the coiled wire extends within the first coil portion.

12. The endovascular device of claim 11,
wherein a plurality of conductor wires extend within the first coil portion, each of the plurality of conductor wires electrically coupled to a respective electrode of the plurality of electrodes, and
wherein the coiled wire extends alongside the plurality of conductor wires within the first coil portion.

13. The endovascular device of claim 1,
wherein the first coil portion includes a polymer, and
wherein the second coil portion does not include the polymer.

14. The endovascular device of claim 1,
wherein the coiled wire includes one or more of platinum-iridium, nickel-cobalt, titanium-tantalum-tin, platinum-tungsten, and beta-titanium alloys.

15. The endovascular device of claim 1, wherein the coil structure is configured to transform from a relatively low-profile delivery configuration to a deployed configuration to position the plurality of electrodes to deliver electrical stimulation to tissue of the patient or sense a patient parameter from a location within the vasculature of the patient.

16. A method comprising:
introducing an endovascular device into vasculature of a patient, the endovascular device comprising:
an elongated body configured to be introduced into the vasculature of the patient, and
a coil structure at a distal portion of the elongated body, the coil structure including:
a first coil portion configured to carry a plurality of electrodes, and
a second coil portion formed from a coiled wire such that the second coil portion together with the coiled wire defines a coiled coil, the coiled wire extending distally of a distal end of the first coil portion and configured to anchor the elongated body and the coil structure within the vasculature of the patient; and
advancing the endovascular device until the plurality of electrodes are at or near a target location in the vasculature of the patient.

17. The method of claim 16,
wherein:
the first coil portion defines a first pitch and a first coil diameter,
the second coil portion defines a second pitch and a second coil diameter, and
the coiled wire defines a third pitch and a third coil diameter,
wherein the third pitch is less than the first pitch and less than the second pitch, and
wherein the third coil diameter is less than the first coil diameter and the second coil diameter.

18. The method of claim 17, wherein the first pitch and the second pitch are the same.

19. The method of claim 17, wherein the first pitch and the second pitch are different.

20. The method of claim 19, wherein the second coil diameter is equal to or greater than the first coil diameter.
